(19) United States
(12) Patent Application Publication
(10) Pub. No.: US 2006/0062797 A1
(43) Pub. Date: Mar. 23, 2006

(54) Novel materials and methods for the treatment of Alzheimer's disease patients

(75) Inventor: Steven O. Smith, St. James, NY (US)

Correspondence Address: Peter G. Carroll;MEDLEN & CARROLL, LLP, Suite 350, 101 Howard Street, San Francisco, CA, 94105, US

(73) Assignee: The Research Foundation of State University of New York at Stony Brook

(21) Appl. No.: 10/945,751

(22) Filed: Sep. 21, 2004

(51) Int. Cl. A61K 39/00; C07K 14/47

(52) U.S. Cl. 424/184.1; 530/350

(57) ABSTRACT

The invention generally relates to the treatment of patients with Alzheimer's disease, at risk for Alzheimer's disease, and other neurodegenerative diseases. More specifically, embodiments of the present invention contemplate novel compositions and methods for the treatment of such patients. In one embodiment, the present invention contemplates proteins and protein variants that function by depolymerizing or preventing the polymerization of amyloid plaques believed to play a role in the etiology of Alzheimer's disease.

This invention was made with funding from the National Institutes of Health, grant number NIH GM46732. Consequently, the United States government has certain rights in this invention.

### FIELD OF THE INVENTION

The invention generally relates to the treatment of patients with Alzheimer's disease, patients at risk for Alzheimer's disease, and other neurodegenerative diseases.

Embodiments of the present invention contemplate novel materials and methods for the treatment of such patients. In one embodiment, the present invention contemplates proteins and protein variants that function by decreasing the presence or formation of amyloid fibrils, protofibrils and soluble oligomers that give rise to amyloid plaques.

### BACKGROUND

Alzheimer's disease is a disorder that destroys cells in the brain. The disease is the leading cause of dementia, a condition that involves gradual memory loss, decline in the ability to perform routine tasks, disorientation in learning, loss of language skills, impairment of judgment and personality changes. As the disease progresses, people with Alzheimer's become unable to care for themselves. The loss of brain cells eventually leads to the failure of other systems in the body. The rate of progression of Alzheimer's varies from person to person. The time from the onset of symptoms until death ranges from 3 to 20 years. The Alzheimer's Association estimates that 4.5 million Americans currently have Alzheimer's disease and that 13.2 million Americans will have the disease by the year 2050 if a method of preventing the disease or curing the disease is not found.

There are currently four drugs approved by the U.S. Food and Drug Administration for treating Alzheimer's disease symptoms. They are tacrine, donepezil, rivastigmine and galantamine. These drugs only treat symptoms of Alzheimer's and do not treat causative events or agents. Additionally, only about one half of the people taking these medications show an improvement in memory and thinking skills and this improvement, moreover, is frequently only a modest and temporary one. Thus, the currently available treatments are inadequate in a very large number of Alzheimer's cases.

What is needed are new materials and methods for the treatment of Alzheimer's disease patients and patients at risk for Alzheimer's disease.

### SUMMARY OF THE INVENTION

The invention generally relates to the treatment of patients with Alzheimer's disease, patients at risk for Alzheimer's disease, and other neurodegenerative diseases. Embodiments of the present invention contemplate novel materials and methods for the treatment of such patients. In one embodiment, the present invention contemplates proteins and protein variants that function by decreasing the presence or formation of amyloid fibrils, protofibrils and soluble oligomers that give rise to amyloid plaques.

It is not intended that the present invention be limited by the nature of the protein inhibitor that inhibits, alleviates, or ameliorates amyloid plaque formation (i.e., for example, by polymerization). Such protein inhibitors can be identified functionally by simply adding them in vitro to amyloid fibrils and measuring the extent of clearing of the fibrils.

In one embodiment, the present invention contemplates an isolated synthetic protein comprising amino acid SEQ ID NO: 1, and variants thereof, wherein said variants are selected from the group consisting of SEQ ID NOs:5-39. In one embodiment, the isolated protein comprises a protease-resistant protecting group.

In one embodiment, the present invention contemplates an isolated synthetic protein comprising amino acid SEQ ID NO:2 and variants thereof, wherein said variants are selected from the group consisting of SEQ ID NOs:40-74. In one embodiment, the isolated protein further comprises a protease-resistant protecting group.

In one embodiment, the present invention contemplates an isolated synthetic protein comprising amino acid SEQ ID NO:3 and variants thereof, wherein said variants are selected from the group consisting of SEQ ID NOs:75-86. In one embodiment, the isolated protein further comprises a protease-resistant protecting group.

In one embodiment, the present invention contemplates an isolated synthetic protein comprising amino acid SEQ ID NO:4 and variants thereof, wherein said variants are selected from the group consisting of SEQ ID NOs:87-98. In one embodiment, the isolated protein further comprises a protease-resistant protecting group.

In one embodiment, the present invention contemplates an isolated synthetic protein comprising a repeating core sequence having an amino terminus and a carboxyl terminus comprising at least two protein binding amino acids (Xaa1 and Xaa2). In one embodiment, the protein binding amino acid Xaa1 has a small amino acid side chain and is selected from the group comprising glycine, alanine, and serine. In one embodiment, the protein binding amino acid Xaa2 has a large hydrophobic or aromatic amino acid side chain and is selected from the group comprising phenylalanine, valine, leucine, isoleucine, methionine, tryptophan and tyrosine. In one embodiment, said core sequence repeats between 1 to 100 times. In one embodiment, said core sequence further comprises additional amino acids added to said amino terminus. In another embodiment, said core sequence further comprises additional amino acids added to said carboxyl terminus. In yet another embodiment, said core sequence further comprises additional amino acids added to said carboxyl terminus and said amino terminus. In one embodiment, the protein further comprises a protease-resistant protecting group. In one embodiment, said core sequence is selected from the group consisting of SEQ ID NO:99-SEQ ID NO: 146.

In one embodiment, the present invention contemplates a composition comprising an Aβ_{1 42} fibril comprising at least one phenylalanine amino acid residue and an isolated synthetic protein comprising a carboxyl terminus selected from the group consisting of SEQ ID NOs: 1-4 and 19, wherein said protein carboxyl terminus binds to said fibril phenylalanine residue. In one embodiment, the protein has amyloid plaque dissolving activity. In one embodiment, the protein comprises a D-amino acid. In one embodiment, the protein comprises a protease resistance group. In one embodiment, the protein comprises a protecting group. In one embodiment, the protein is N-terminally acetylated and C-terminally amidated. In one embodiment, the protein comprises at least five additional N-terminal amino acids and C-terminal amino acids.

In one embodiment,. the present invention contemplates a method, comprising:

a) providing; i) an amyloid fibril inhibitor protein comprising a core sequence, wherein said core sequence comprises a first and second protein binding amino acid residue and at least one polar amino acid residue; ii) a patient comprising a peptide fibril; b) contacting said inhibitor protein to said fibril under conditions such that said fibril depolymerizes.

In one embodiment, the fibril results from Alzheimer's disease. In one embodiment, the fibril results from Parkinson's disease.

In one embodiment, the present invention contemplates a method, comprising:

a) providing; i) an amyloid fibril inhibitor protein comprising a core sequence, wherein said core sequence comprises a first and second protein binding amino acid residue and at least one polar amino acid residue; ii) a patient at risk for peptide fibril formation;

c) administering said inhibitor protein to said patient under conditions such that prevent polymerization of said peptide fibril in said patient.

In one embodiment, the present invention contemplates a method of treating Alzheimer's disease, comprising: a) providing: i) a subject having symptoms of Alzheimer's disease and, ii) a composition of matter comprising a protein having a core sequence, wherein said core sequence comprises a first and second protein binding amino acid residue and at least one polar amino acid residue; and b) administering said composition to said subject until said symptoms are reduced. In one embodiment, the protein comprises from between four and five hundred amino acids. In one embodiment, the protein is selected from a group consisting of SEQ ID NO: 1, SEQ ID NO:2, SEQ ID NO:3, SEQ ID NO:4 and variants thereof. In one embodiment, the amino acid terminus is blocked with an acetyl group and said carboxyl terminus is blocked with an acetate group. In one embodiment, the protein further comprises at least one non-naturally occurring amino acid. In one embodiment, the protein further comprises at least one D-amino acid.

In one embodiment, the present invention contemplates a method, comprising: a) providing; i) an amyloid fibril inhibitor protein or peptide; ii) a patient comprising a peptide fibril or oligomer; and b) contacting said inhibitor protein or peptide to said fibril under conditions such that said fibril depolymerizes or oligomers are blocked from forming fibrils. In one embodiment, said fibrils or oligomers result from Alzheimer's disease. In another embodiment, said fibrils or oligomers result from Parkinson's disease. In another embodiment, said fibrils or oligomers result from the prion protein associated with transmissible spongiform encephalopathies.

In one embodiment, the present invention contemplates a method, comprising: a) providing; i) an amyloid fibril inhibitor protein or peptide; ii) a patient at risk for peptide fibril or oligomer formation; and b) administering said inhibitor protein or peptide to said patient under conditions such that prevent polymerization of said fibrils or oligomers in said patient.

In one embodiment, the present invention contemplates a composition comprising the amino acid sequence RGTFQGKF (SEQ ID NO:1). The present invention is not, however, limited to SEQ ID NO: 1. In one embodiment, SEQ ID NO: 1 comprises additional amino acids added to the amino terminus, to the carboxyl terminus or to both the amino and carboxyl termini. The present invention is not limited to the number of amino acids added to the amino or carboxyl termini or the sequence of amino acids added to the amino or carboxyl termini. In one embodiment, from one to about 500 amino acids, and more preferably from about 10-50 amino acids are added to the amino terminus. In another embodiment, from one to about 500 amino acids, and more preferably from about 10-50 amino acids, are added to the carboxyl terminus. In yet another embodiment, from one to about 500 amino acids, and more preferably from about 10-50 amino acids, are added to both the amino and carboxyl termini. In one embodiment, the number of amino acids added to both the amino and carboxyl termini is the same. In another embodiment, the number of amino acids added to the amino and carboxyl termini is different. In still yet another embodiment, the present invention contemplates that any of the amino acids may be D-amino acids or L-amino acids.

In one embodiment, the present invention contemplates a composition comprising a SEQ ID NO: 1 variant. In one embodiment, a variant of the sequence RGTFEGKF-NH₂ (SEQ ID NO: 1) is selected from the group comprising RFTGEFKG-NH₂ (SEQ ID NO:2), RFTGEF-NH₂ (SEQ ID NO:3) or RFTAEF-NH₂ (SEQ ID NO:4). Other exemplary sequences that are SEQ ID NO: 1 variants are presented in Table 1. In another embodiment, SEQ ID NOs: 2 - 4 comprise additional amino acids added to the amino terminus, to the carboxyl terminus or to both the amino and carboxyl termini. The present invention is not limited to the number of amino acids added to the amino or carboxyl termini or the sequence of amino acids added to the amino or carboxyl termini.

In one embodiment, from one to about 500 amino acids, and more preferably from about 10-50 amino acids are added to the amino terminus. In another embodiment, from one to about 500 amino acids, and more preferably from about 10-50 amino acids are added to the carboxyl terminus. In yet another embodiment, from one to about 500 amino acids, and more preferably from about 10-50 amino acids are added to both the amino and carboxyl termini. In one embodiment, the number of amino acids added to both the amino and carboxyl termini are the same. In another embodiment, the number of amino acids added to the amino and carboxyl termini is different. In still yet another embodiment, the present invention contemplates that any of the amino acids may be D-amino acids or L-amino acids. Non-limiting examples of peptides of the present invention are given in Table 1 and Table 2. In another embodiment, sequences that are at least 90% homologous to the sequences of SEQ ID NOs: 1 - 4 and are effective in dissolving or preventing the formation of amyloid plaques contemplated by the present invention.

In one embodiment, the present invention contemplates several general amino acid formulae comprising amyloid fibril inhibitor protein sequences including, but not limited to, SEQ ID NO: 1 and variants thereof. In various embodiments, SEQ ID NO: 1 variants may be represented in the following general formulae as non-limiting examples:

i) (Xaa4)a-(Xaa2-Xaa3-Xaa1)ₙ-(Xaa4)_{b}; ii) (Xaa4)ₐ-(Xaa3-Xaa2-Xaa3-Xaa1-Xaa3-Xaa1)ₙ-(Xaa4)_{b}; iii) (Xaa4)ₙ-(Xaa3-Xaa2-Xaa3-Xaa2-Xaa3-Xaa1)ₙ-(Xaa4)_{b}: iv) (Xaa4)ₙ-(Xaa3-Xaa1-Xaa3-Xaa2)ₙ-(Xaa4)_{b}; v) (Xaa4)ₐ-(Xaa3-Xaa1-Xaa3-Xaa1-Xaa3-Xaa2)ₙ-(Xaa4)_{b}; vi) (Xaa4)ₐ-(Xaa3-Xaa1-Xaa3-Xaa2-Xaa3-Xaa2)ₙ-(Xaa4)_{b}; and vii) (Xaa4)ₐ-(Xaa2-Xaa2-Xaa3-Xaa1-Xaa1-Xaa3)ₙ-(Xaa4)_{b} wherein Xaa1 is a small side chain amino acid protein binding residue (i.e., for example, a hydrogen, a hydroxyl, or a methyl group), Xaa2 is a large side chain amino acid protein binding residue (i.e., for example, a hydrophobic or an aromatic group, such as, isoleucine or tyrosine), Xaa3 is a polar or β-branched amino acid and Xaa4 is any amino acid, and further wherein subscripts a and b indicate a number from zero to 500 and, more preferably, from zero to 50. In one embodiment, n is a number from 1 to about 100. In a preferred embodiment, n is less than 20. In a more preferred embodiment, n is 2. In another embodiment, n includes a fractional amount (e.g., n may equal 1½ which means, for example, that the formulae (i-vii) would comprise six amino acid residues). Also, for example, n may equal 1¼, ¾, 2½, 2⅙, 2¾, 5⅚, 10¼, etc., as long as the final number of amino acid residues is a whole number. The present invention is not limited to which end of the sequence the fractional part of the sequence is taken from. For example, one or more amino acids may be copied from the amino or from the carboxyl termini and added to the respective termini (i.e., the amino acid(s) copied from the amino terminus would be added to the amino terminus and the amino acid(s) copied from the carboxyl terminus would be copied to the carboxyl terminus). Likewise, in another embodiment, the amino acids may be copied from both the amino and carboxyl termini and added to the respective termini (i.e., the amino acids copied from the amino terminus would be added to the amino terminus and the amino acids copied from the carboxyl terminus would be copied to the carboxyl terminus). Furthermore, amino acids copied for the amino terminus may be copied to the carboxyl terminus and amino acids copied from the carboxyl terminus may be copied to the amino terminus.

The present invention is not limited to which end of the core sequence an additional fractional sequence portion is added. In the example above, for instance, a core sequence might comprise three amino acids (i.e., for example, as in general formula (i)) and optionally, at least one left (Xaa4ₐ) or at least one right (Xaa4_{b}) amino acids. It is also noted that the present invention is not limited to the specific sequences or general formulae given here. Any peptide sequence that solubilizes or dissolves amyloid plaques is contemplated by the present invention. In another embodiment of the present invention, any peptide sequence that solubilizes or dissolves amyloid plaques and comprises polar amino acids is contemplated by the present invention. In another embodiment of the present invention, the core sequences based on the general formulae (i)-(vii) may be used in reverse. For example, the core sequence of general formula (vii) comprising (Xaa2-Xaa2-Xaa3-Xaa1-Xaa1-Xaa3)n, may also be read (Xaa3-Xaa1-Xaa1-Xaa3-Xaa2-Xaa2)n. For exemplary variant core sequences based on the general formulae (i)-(vii), see Table 2.

In one embodiment, an amyloid fibril inhibitor protein comprises at least one Xaa3 amino acid residue that is capable of solubilizing a target protein. In one embodiment, the Xaa3 amino acid residue comprises at least one polar amino acid residue. In another embodiment, the Xaa3 amino acid residue comprises at least one ionically charged amino acid residue. In another embodiment, the Xaa3 amino acid residue comprises at least 10% of the total number of polar amino acids in the inhibitor protein. In another embodiment, the Xaa3 amino acid residue comprises at least 50% of the total number of polar amino acids in the inhibitor protein. In one embodiment, the Xaa3 amino acid residue comprises at least two different polar amino acids. In another embodiment, the Xaa3 amino acid residue comprises the same amino acid. In one embodiment, the Xaa3 amino acid residue comprises any combination of polar amino acids that is capable of solubilizing a target protein (e.g., for example, amyloid fibrils).

In one embodiment, Xaa1 comprises small side chain naturally occurring amino acids including, but not limited to, glycine (G), alanine (A), or serine (S). In one embodiment, Xaa1 comprises the same amino acid. In another embodiment, Xaa1 comprises different amino acids. In yet another embodiment, Xaa1 comprises non-naturally occurring amino acids. In one embodiment, the small side chain Xaa1 amino acids comprise protecting groups. In one embodiment, the protecting groups may be selected from the group comprising Fmoc-L-allylglycine, Boc-L-allylglycine dicyclohexylammonium salt, Fmoc-D-allylglycine, Boc-D-allylglycine dicyclohexylammonium salt. In another embodiment, the Xaa1 amino acids do not comprise protecting groups. In another embodiment, non-naturally occurring Xaa1 amino acids are contemplated (AnaSpec; San Jose, Calif.).

In one embodiment, Xaa2 comprises large side chain naturally occurring amino acids including, but not limited to, phenylalanine (F), tyrosine (Y), tryptophan (W), valine (V), leucine (L), or isoleucine (I). In one embodiment, Xaa2 comprises the same amino acid. In another embodiment, Xaa2 comprises different amino acids. In yet another embodiment, the large side chain Xaa2 amino acids are non-naturally occurring amino acids. In one embodiment, the large side chain Xaa2 amino acids comprise 10 protecting groups. In one embodiment, the protecting groups may be selected from the group comprising Fmoc-DL-methionine methylsulfonium chloride, Boc-DL-methionine methylsulfonium chloride, Fmoc-a-methyl-DL-methionine, Boc-a-methyl-DL-methionine, Fmoc-L-selenomethionine, Boc-L-selenomethionine, Fmoc-DL-selenomethionine or Boc-DL-selenomethionine. In another embodiment, the Xaa2 amino acids do not comprise protecting groups. In another embodiment, non-naturally occurring Xaa2 amino acids are contemplated (AnaSpec; San Jose, Calif.).

In one embodiment, Xaa3 comprises polar, ionically charged, or β-branched naturally occurring amino acids including, but not limited to, histidine (H), lysine (K), arginine (R), aspartic acid (D), glutamic acid (E) and amides thereof, asparagine (N), and glutamine (Q). In yet another embodiment, Xaa3 amino acids comprise non-naturally occurring polar, ionically charged, or β-branched amino acids. In one embodiment, the polar, ionically charged, or P-branched Xaa3 amino acids comprise protecting groups. In one embodiment, an Xaa3 amino acid protecting group may be selected from the group comprising Fmoc-(Boc-4- aminomethyl)-L- phenylalanine, Boc-(Fmoc-4-aminomethyl)-L-phenylalanine, Fmoc-(Boc-4- aminomethyl)-D-phenylalanine, Boc-(Fmoc-4-aminomethyl)-D- phenylalanine, Fmoc-4-amino-L-phenylalanine, Boc-4-amino-L-phenylalanine, Fmoc-4-amino-D- phenylalanine, Boc-4-amino-D-phenylalanine, Fmoc-(Boc-4-amino)- L-phenylalanine, Fmoc-(Boc-4-amino)-D-phenylalanine, Fmoc-4-bromo- L-phenylalanine, Boc-4-bromo-L-phenylalanine, Fmoc-4-bromo-D-phenylalanine, Boc-4-bromo- D-phenylalanine, Fmoc-4-bis(2-chloroethyl)amino-L-phenylalanine, Boc-4- bis(2-chloroethyl)amino-L-phenylalanine, Fmoc-2-chloro-L-phenylalanine, Boc-2-chloro-L-phenylalanine, Fmoc-4,5-dehydro-L-leucine or Boc-4,5-dehydro- L-leucine. In another embodiment, the polar, ionically charged, or branched Xaa3 amino acids may also be used without protecting groups. In another embodiment, non-naturally occurring polar, ionically charged or β-branched Xaa3 amino acids are contemplated (AnaSpec; San Jose, Calif.).

In one embodiment, an amyloid fibril inhibitor protein comprises Xaa1 and Xaa2 amino acid residues, wherein only a portion of the Xaa1 and Xaa2 amino acid residues comprise peptide binding residues. In one embodiment, Xaa1 and Xaa2 amino acid residues comprise peptide binding residues sufficient for attachment to a target protein.

In one embodiment, the Xaa1 and Xaa2 amino acid residues comprise at least 10% of the total number of peptide binding amino acids in the inhibitor protein. In another embodiment, the Xaa1 and Xaa2 amino acid residues comprise at least 50% of the total number of peptide binding amino acids in the inhibitor protein. In one embodiment, Xaa1 and Xaa2 amino acid residues are different amino acids. In another embodiment, the Xaa1 and Xaa2 amino acid residues are the same amino acid. In one embodiment, Xaa1 and Xaa2 comprise any combination of amino acids that permit the binding of an amyloid fibril inhibitor protein to a target protein (e.g., for example, amyloid fibrils).

In one embodiment, the present invention contemplates amyloid fibril inhibitor proteins terminating with a CONH₂ group at the carboxyl terminus. Although the present invention is not limited to any particular theory, it is believed that the CONH₂ group at the carboxyl terminus reduces degradation of the protein. In another embodiment, the inhibitor protein terminates with a COOH group at the carboxyl terminal. In yet another embodiment, the inhibitor protein terminates with an NH2 group at the amino terminus. In another embodiment, the inhibitor protein terminates with a diamino group (i.e., NH₂-NH₂) at the amino terminus. In another embodiment, the inhibitor protein further comprises at least one carbohydrate group.

It is not intended that the present invention be limited to specific amyloid fibril inhibitor proteins. In one embodiment, the present invention contemplates amyloid inhibitor proteins that comprise peptides that are protease resistant. In one embodiment, the protease-resistant peptides comprise protecting groups. In another embodiment, the endoprotease-resistant peptides comprise at least one D-amino acid. In another embodiment, the present invention contemplates an inhibitor protein comprising the amino acid sequence RGTFQGKF (SEQ ID NO: 1), or a variant thereof, comprising an N-terminal acetylation (“Ac”) and/or a C-terminal amidation (“NH₂”) for exoproteinase protection. In one embodiment, the inhibitor protein comprises Ac-XaaRGTFQ-GKFXaa-NH₂ (SEQ ID NO: 156) wherein Xaa is any amino acid, and further wherein the number of additional amino acids may vary from between 0 and 100, thereby imparting a resistance to proteolysis. In one embodiment, SEQ ID NO: 156 has improved bioavailability following in vivo administration.

In another embodiment, the present invention also contemplates amyloid fibril inhibitor proteins comprising D-amino acids thereby protecting the protein from endoprotease degradation. In one embodiment, the D-amino acid substitutes for a pre-existing L-amino acid. In another embodiment, the D-amino acid is an additional amino acid (i.e., for example, as an Xaa4 amino acid residue). It is not intended that the present invention be limited to particular amino acids and, in particular, D-isomers. In one embodiment, a D-amino acid comprises all amino acids having stereoisomeric forms. Although it is not necessary to understand the mechanism of an invention, it is believed that stereoisomeric amino acid forms are mirror-image structures, and by convention, are differentiated as D and L forms. It is further believed that stereoisomeric forms cannot be interconverted from a D to L (or L to D) form without breaking a chemical bond. One having skill in the art would recognize that, with rare exceptions, only the L forms of amino acids are found in naturally occurring proteins.

In one embodiment, the present invention contemplates RGTFQGXaaF (SEQ ID NO: 164)-containing peptides for treatment of Alzheimer's patients and patients at risk for Alzheimer's disease (i.e., where “Xaa” represents a lysine D-isomer). In another embodiment, any L-amino acid may be substituted with a D-amino acid. In yet another embodiment of the present invention, more than one L-amino acid may be substituted with a D-amino acid. In still yet another embodiment, any peptide contemplated by the present invention (e.g., those exemplified by SEQ ID NOs: 1 - 4, those sequences specifically noted above, those given in Table 1 and Table 2, and other sequences contemplated by the general formulae (i)-(vii) above, may have one or more L-amino acids substituted with a D-amino acid.

In another embodiment, the present invention contemplates a method for treating Alzheimer's disease and other neurodegenerative diseases. In one embodiment, the present invention contemplates a method to dissolve or prevent the formation of amyloid plaques. In another embodiment, the present invention further contemplates that the compositions of the present invention are used in conjunction with other therapeutic drugs, agents or peptides. For example, the compositions of the present invention may be administered with agents that relieve symptoms of Alzheimer's disease or other neurodegenerative diseases (e.g., as discussed above), with agents that relieve pain (e.g., novacain, morphine, aspirin and other medically recognized pain relief agents), with agents that aid in the administration of the compounds of the present invention (e.g., carriers, elixirs, suspension agents, antimicrobial agents, etc.). In yet another embodiment of the present invention, the compositions contemplated by the present invention further comprise excipients and fillers that have no pharmacological activity. Non-active ingredients are, for example, useful for ease of administration, manufacture and packaging. Non-limiting examples of non-active ingredients include saline solutions, antibiotics, thickeners, thinners, oils, colors, pH adjusting agents, flavors, etc.

In another embodiment, the present invention contemplates that compositions of the present invention are used for assaying for non-peptidic compounds that, at least partially, inhibit formation of, or dissolve, amyloid fibril plaques. In one embodiment, the present invention contemplates a method for testing a non-peptidic compound, comprising; a) providing: i) a parent peptide having the sequence set forth in the sequences of the present invention (e.g., SEQ ID NOs: 1 - 4); ii) a candidate non-peptidic mimetic of said parent peptide; iii) a solution comprising amyloid fibrils; b) adding said candidate mimetic to said solution under conditions wherein said parent peptide inhibits amyloid fibril plaque formation; and c) detecting amyloid fibril plaque formation inhibition by said candidate mimetic.

### BRIEF DESCRIPTION OF THE FIGURES

FIG. 1A shows an exemplary amino acid sequence for Amyloid Precursor Protein (APP) (i.e., Asp⁶⁷² to Lys⁷²⁴). In one embodiment, an APP fragment comprises an amyloid fibril peptide (i.e., Aβ₁₋₄₂) that corresponds to Asp⁶⁷² to Ala⁷¹³ (SEQ ID NO: 157).

Note that Gly²⁵ is adjacent to amino acids having a high propensity to form a β-turn. Further, Gly³³, Gly³⁷ and Gly³⁸ are found in the context of hydrophobic and β-branched amino acids. (Note: Read upper strand right-left and lower strand left-right). Boxed Region: ¹³C-Cross Peak.

FIG. 1B shows one embodiment of a glycophorin A (GpA) amino acid sequence (i.e., Glu⁷⁰ to Leu⁹⁸:SEQ ID NO: 163). Truncating the sequence at Gly⁸⁶ mimics the hydrophobic C-terminus of Aβ₁₋₄₂ resulting in an amyloid fibril peptide corresponding to Glu⁷⁰-Glu⁸⁶ (SEQ ID NO: 158). Boxed Region: ¹³C-Cross Peak.

FIG. 1C shows one embodiment of an amyloid fibril peptide comprising the prion protein amino acid sequence (i.e., Ala¹¹⁸ to Ser¹³⁵ SEQ ID NO: 159).

FIG. 2A shows exemplary infrared spectra of the GpA amide I region. Curve α: a full length GpA (SEQ ID NO: 163). Curve P: a truncated GpA (SEQ ID NO: 158). All peptides were reconstituted with dimyristoylphosphatidylcholine (DMPC).

FIG. 2B illustrates an exemplary electron micrograph of fibrils formed by the truncated GpA₇₀₋₈₆ peptide (SEQ ID NO: 158).

FIG. 2C illustrates an exemplary electron diffraction pattern of fibrils formed by the truncated GpA₇₀₋₈₆ peptide (SEQ ID NO: 158).

FIG. 3 shows one embodiment of a potential b-sheet packing arrangement using, for example, GpA₇₀₋₈₆ fibrils (i.e., for example, dovetail packing). This space-filling molecular model demonstrates parallel, in-register stacking of β-strands within a β-sheet creating long channels at the position of glycine. The channels run in the direction of the fibril axis (out of the page). In this example, glycine's small side chain (left side fibril indicated by single arrow) creates a surface groove that facilitates tight dovetail packing with large hydrophobic side chains on the opposite β-sheet (right side fibril indicated by double arrow). The two β-sheets are separated from one another for clarity.

FIG. 4A shows exemplary data of a two dimensional solid-state NMR spectra from GpA⁷⁰⁻⁸⁶ (SEQ ID NO: 158) fibrils formed from an equimolar mixture of 1-¹³C-Gly⁷⁹, 2-¹³C-Gly⁸³-labeled peptide and 2-¹³C-Gly⁷⁹, 1-¹³C-Gly⁸³-labeled peptide. The cross peaks in boxes which occur between the diagonal peaks [indicated by arrows] corresponding to the 1-¹³C and 2-¹³C resonances show that the peptides have a parallel, in-register orientation of the peptides within a β-sheet. X-Axis: ¹³C Chemical Shift. Y-Axis: ¹³C Chemical Shift.

FIG. 4B shows exemplary data of a two dimensional solid-state NMR spectra from GpA₇₀₋₈₆ (SEQ ID NO: 158) fibrils formed from an equimolar mixture of ¹³CH₃ Met⁸¹-labeled peptide and 1-¹³C-Gly⁷⁹-labeled peptide. The cross peak (boxed) which occurs between the diagonal peak (indicated by an arrow) corresponding to the Gly⁷⁹ resonance and the diagonal peak (not shown) corresponding to Met⁸¹ is consistent with dovetail sheet-to-sheet packing. The top trace represents a one-dimensional slice through the two dimensional spectrum at the position of the dashed line. X-Axis: ¹³C Chemical Shift. Y-Axis: ¹³C Chemical Shift.

FIG. 4C shows exemplary data of a two dimensional solid-state NMR spectra from GpA⁷⁰⁻⁸⁶ (SEQ ID NO: 158) fibrils containing only the ¹³CH₃ Met⁸¹-labeled peptide. The carbonyl signal is less intense here because only natural abundance ¹³C signal is observed. The top trace represents a one-dimensional slice through the two dimensional spectrum at the position of the dashed line. X-Axis: ¹³C Chemical Shift. Y-Axis: ¹³C Chemical Shift.

FIG. 4D shows exemplary data of a two dimensional solid-state NMR spectra from GpA₇₀₋₈₆ (SEQ ID NO: 158) fibrils formed from an equimolar mixture of ¹³CH₃ Met81-labeled peptide and 1-₁₃C-Gly⁷⁹-labeled peptide in the presence of an equimolar amount of I1 (SEQ ID NO:1). The cross peak observed in FIG. 4B between Met⁸¹ and Gly⁷⁹ is absent. The top trace represents a one-dimensional slice through the two dimensional spectrum at the position of the dashed line. X-Axis: ¹³C Chemical Shift. Y-Axis: ¹³C Chemical Shift.

FIG. 5 shows exemplary data of a two dimensional solid-state NMR spectra from Aβ₁₋₄₂ (SEQ ID NO: 157) fibrils formed from an equimolar mixture of ¹³CH₃ Met³⁵-labeled peptide and a 2-¹³C-Gly³⁷-labelled peptide. The cross peaks in boxes which occur between the diagonal peaks (indicated by arrows) corresponding to the Met³⁵ and Gly³⁷ resonances are consistent with dovetail sheet-to-sheet packing. X-Axis: ¹³C Chemical Shift. Y-Axis: ¹³C Chemical Shift.

FIG. 6 presents exemplary data showing that SEQ ID NO:1 (I1) inhibits fibrillization of Aβ₁₋₄₂. X Axis: Incubation Day. Y Axis: Fluorescence Intensity. Squares: Aβ₁₋₄₂ peptides. Circles: Aβ₁₋₄₂ peptides and II at a molar ratio of 1:20.

FIG. 7 presents exemplary data showing the dose-dependent inhibition kinetics of SEQ ID NO:1 (I1) or SEQ ID NO:160 (I6). X-Axis: Vertical Striped Bar: Aβ₁₋₄₂ peptide. Open Bar: 1:1 molar ratio Aβ₁₋₄₂ peptide:I1. Crosshatched Bar: 1:10 molar ratio Aβ₁₋₄₂ peptide:I1. Stippled Bar: 1:20 molar ratio Aβ₁₋₄₂ peptide:I1. Horizontal Striped Bar: 1:20 molar ratio Aβ₁₋₄₂ peptide:I6. Y-Axis: Fluorescence Intensity (%).

FIG. 8 presents exemplary time course data showing that SEQ ID NO:1 (I1) depolymerizes Aβ₁₋₄₂ fibrils. X-Axis: Incubation Day. Y-Axis: Fluorescence Intensity (%).

FIG. 9 shows exemplary time course data demonstrating that, in an in vitro neuronal cell culture, Aβ₁₋₄₂ peptide is toxic. Y-Axis: Cell death percentage. X-Axis:

Treatment Day. Top curve (preincubated): Aβ₁₋₄₂ peptide added to neuronal cell culture after amyloid fibril polymerization. Bottom curve (not preincubated): Aβ₁₋₄₂ peptide added to neuronal cell culture before amyloid fibril polymerization.

FIG. 10A provides exemplary data showing that SEQ ID NO:1 (I1) does not inhibit Aβ₁₋₄₂ peptide induced neurotoxicity after a 24 hour incubation. CT: Control cell culture. Open Bar: Aβ₁₋₄₂ peptide. Crosshatched Bar: 1:10 Aβ₁₋₄₂ peptide+I1. Stippled Bar: 1:20 Aβ₁₋₄₂ peptide+I1.

FIG. 10B provides exemplary data showing that SEQ ID NO:160 (I6) does not inhibit Aβ₁₋₄₂ peptide induced neurotoxicity after a 24 hour incubation. CT: Control cell culture. Open Bar: Aβ₁₋₄₂ peptide. Crosshatched Bar: 1:10 Aβ₁₋₄₂ peptide+16. Stippled Bar: 1:20 Aβ₁₋₄₂ peptide+I6.

FIG. 10C provides exemplary data showing that SEQ ID NO:1 (I1) does inhibit Aβ₁₋₄₂ peptide induced neurotoxicity after a 48 hour incubation. CT: Control cell culture. Open Bar: Aβ₁₋₄₂ peptide. Crosshatched Bar: 1:10 Aβ₁₋₄₂ peptide+I1. Stippled Bar: 1:20 Aβ₁₋₄₂ peptide+I1.

FIG. 10D provides exemplary data showing that SEQ ID NO:160 (I6) does not inhibit Aβ₁₋₄₂ peptide induced neurotoxicity after a 48 hour incubation. CT: Control cell culture. Open Bar: Aβ₁₋₄₂ peptide. Crosshatched Bar: 1:10 Aβ₁₋₄₂ peptide+I6. Stippled Bar: 1:20 Aβ₁₋₄₂ peptide+16.

### DEFINITIONS

The term, “fibrillization”, as used herein, refers to any process where peptides come or bind together (i.e., for example, by polymerization or aggregation) and form a “plaque” (e.g., amyloid peptide fibrils).

The term “plaque”, as used herein, refers to any association of amyloid proteins.

Such associations may comprise “an aggregate” representing a loose association (i.e., that having no order) or “a fibril” representing an organized association having long range order and orientation (i.e., that having order).

The term “fibril”, as used herein, refers to any polymerized or aggregated peptide composition. For example, “an amyloid plaque” or “amyloid fibril” comprises polymerized or aggregated Aβ₁₋₄₂ peptides.

The term “protein binding residue”, as used herein, refers to any amino acid that is capable of attaching to another amino acid. The attachment may be covalent, electrostatic, ionic, hydrophobic, Van der Waals interactions or any combination thereof.

The term “hydrophilic”, as used herein, refers to any compound that attracts ionic and/or strongly polar groups and readily interacts with water.

The term “polar group”, as used herein, refers to any chemical grouping in which the distribution of electrons creates a molecular dipole moment, thereby enabling the molecule to take part in electrostatic interactions, including interactions with water. The term “polar amino acid” shall refer to an amino acid with polar groups that readily interact with water.

The term “protein binding amino acid”, as used herein, refers to any amino acid that can attach to any portion of another protein or glycoprotein.

The term “non-naturally occurring amino acid”, as used herein, refers to any amino acid that is not normally found in living organisms.

The term “D-amino acid”, as used herein, refers to any amino acid that is a dextro-rotatory stereoisomer, as opposed to a levo-rotatory stereoisomer or L-amino acid. The D-amino acid may be a stereoisomer of a naturally occurring or a non-naturally occurring amino acid.

The term “amino terminus” or “amine terminus”, as used herein, refers to any end of a peptide or protein comprising an NH₂ group.

The term “carboxyl terminus”, as used herein, refers to the end of a peptide or protein comprising a COOH group.

The term “protease resistant”, as used herein, refers to any peptide or protein that comprise a tertiary or quaternary structure that interferes with protease attachment and subsequent degradation.

The term “protease”, as used herein, refers to any enzyme that catalyses the splitting of interior peptide bonds (i.e., for example, an endoprotease) or terminal peptide bonds (i.e., for example, an exoprotease) in a protein.

The term “protecting group”, as used herein, refers to any molecule that is added to a molecule for the purpose of providing degradation protection. For example, peptides or proteins may have protecting groups for protection against proteases. Protecting groups may include, but are not limited to, N-terminal acetyl groups and C-terminal acetate groups. It is not intended that the present invention be limited to the foregoing protecting groups, in fact, any compatible protecting group may be used.

The term “inhibit amyloid plaque formation”, as used herein, refers to any partial (e.g., for example, 10%) or total (e.g., for example, 100%) inhibition of the formation of amyloid plaques. Such inhibition may dissolve an already formed amyloid plaques by total or partial dissolution and/or depolymerization or prevent the formation of an amyloid plaque by total or partial inhibition of polymerization.

The term “target peptide”, as used herein, refers to any peptide, protein or glycoprotein to which an amyloid fibril inhibitor protein of the present invention is believed to attach (i.e., for example, the Aβ₁₋₄₂ peptide).

The term “core sequence”, as used herein, refers to a specific amino acid sequence motif that specifically binds to a peptide fibril (i.e., for example, an amyloid fibril or amyloid plaque). The core sequence motif may range between two to eight amino acids, but preferably between four to six amino acids, but more preferably between three to five amino acids. The “core sequence” may, or may not be repeated, as represented in general formulae (i)-(vii).

The term “patient” or “subject”, as used herein, refers to any individual having symptoms of, or at risk for, any neurodegenerative disease (e.g., for example, Alzheimer's disease, Parkinson's disease, cystic fibrosis or bovine spongiform encephalopathy). For example, it is known that patients may be at risk for Alzheimer's or Parkinson's disease because of family history (i.e., indicative of genetic predisposition). A patient or subject may be human or non-human and may include, for example, animal strains or species used as “model systems” for research purposes.

The term “dissolving agent”, as used herein, refers to any composition or agent that changes or causes a change from a solid or semi-solid to a more dispersed form.

The term “drug” as used herein, refers to any medicinal substance used in humans or other animals. Drugs may include, but are not limited to, natural or recombinant peptides or proteins and their respective variants and analogs, natural or synthetic molecules and their derivatives and analogs, pharmaceuticals, hormones, antimicrobials, neurotransmitters, etc.

The term “receptor”, as used herein, refers to any structure which recognizes a binding molecule (e.g., a ligand). For example, a receptor may reside on a cell surface which recognizes a neurotransmitter.

The term “antagonist”, as used herein, refers to any molecule or compound which inhibits a biological or biochemical process. For example, an antagonist may inhibit the action or formation of a “native” or “natural” compound, (i.e., for example, amyloid fibrils). Antagonists may or may not be homologous to these natural compounds in respect to conformation, charge or other characteristics. Thus, antagonists may be recognized by the same or different receptors that are recognized by the natural compound.

The term “isolated” and “purified”, as used herein in the context of peptide sequences, refers to any separation of a desired peptide sequence(s) from non-desired peptide sequences and, optionally, other contaminants (e.g. for example, lipids, carbohydrates, nuclei acids, etc.). The terms “isolated” and “purified” are contemplated to mean isolation and purification from between approximately 50% to approximately 100% homogeneity.

The term “synthetic”, as used herein, refers to any non-biological chemical reaction process. A “synthetic” peptide, for instance, may be created using standard condensation organic chemistry using automated peptide synthesizers or combinatorial chemistry including, but not limited to, light-controlled or photolithographic microarrays. Preferably, the inhibitor peptides of the present invention are made synthetically. However, they may also be make recombinantly if so desired.

The term “pharmaceutical composition”, as used herein, comprises any therapeutically active compound. Such a therapeutically active compound may comprise a sequence, or sequences, of the present invention and a carrier and/or an excipient. The terms “protein”, “peptide”, or “polypeptide”, as used herein, refer to any compound comprising amino acids joined via peptide bonds and are used interchangeably. A “protein”, “peptide”, or “polypeptide” amino acid sequence may also comprise post-translational modifications.

The term “amino acid sequence”, as used herein, refers to the primary (i.e., linear) structure of a protein, peptide, or polypeptide.

The term “portion”, as used herein in reference to a protein refers to a fragment of that protein. Protein fragments or portions may range in size from four amino acid residues to the entire amino sequence minus one amino acid.

The term “chimera” or “hybrid”, as used herein, refers to any polypeptide comprising two or more peptide sequences that physically interact. The peptide sequences includes those obtained from the same or from different species or may be synthetic.

The term “fusion”, as used herein, refers to any chimeric polypeptide comprising a protein of interest joined to an exogenous protein fragment (i.e., a fusion partner). The purpose of a fusion partner includes, but is not limited to, enhancing solubility or providing a purification “affinity tag”. If desired, the fusion partner may be removed after or during purification.

The term “homolog” or “homologous”, as used herein, refers to any polypeptide having a high degree of identity to a reference polypeptide between their primary sequence structure, tertiary structure identity, the active site, or the mechanism of action.

For example, a homolog peptide or protein may have a greater than 60 % sequence identity, more preferably greater than 75% sequence identity, and still more preferably greater than 90% sequence identity, with a reference polypeptide. The term “substantial identity”, as used herein, refers to two peptide sequences, when optimally aligned (using, for example, programs such as GAP or BESTFIT) share at least 80% primary sequence structure identity, preferably at least 90% primary sequence structure identity, more preferably at least 95% primary sequence structure identity or more (e.g., for example, 99% sequence identity). Preferably, residue positions which are not identical are related by conservative amino acid substitutions.

The terms “variant” and “mutant”, as used herein when in reference to a polypeptide, refers to an amino acid sequence that differs from another amino acid sequence by one or more amino acids. The variant or mutant may have “conservative” changes, wherein a substituted amino acid has similar structural or chemical properties.

One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant or mutant may have “non-conservative” changes (e.g., replacement of a glycine with a tryptophan). Other variants or mutants may also include amino acid deletions or insertions (i.e., additions), either singly or simultaneously. Guidance in determining which and how many amino acid residues may be substituted, inserted or deleted without abolishing biological activity may be found using computer programs well known in the art, for example, DNAStar software. Variants or mutants can be tested in functional assays. Variants or mutants may comprise less than a 10%, preferably less than a 5% and still more preferably less than a 2% difference in amino acid sequence.

The term “domain”, as used herein, refers to any polypeptide subsection which possesses a unique structural and/or functional characteristic. A domain typically comprises contiguous amino acids, although it may also comprise amino acids which act in concert or which are in close proximity due to folding or other configurations.

The term “at least one symptom is reduced”, as used herein, is not interpreted as a complete absence, or permanent absence, of a symptom. That is, a partial reduction, or temporary reduction, in the symptom is contemplated.

The term “Alzheimer's Disease”, as used herein, refers to one form of dementia that is a progressive, degenerative brain disease which impairs memory, thinking, and behavior. In the early stages, symptoms may be subtle and resemble signs that people mistakenly attribute to “natural aging.” Symptoms initially often include: repeating statements, misplacing items, having trouble finding names for familiar objects, getting lost on familiar routes, personality changes, losing interest in things previously enjoyed, difficulty performing tasks that take some thought, but used to come easily, like balancing a checkbook, playing complex games (such as bridge), and learning new information or routines. In a more advanced stage, symptoms are more obvious: forgetting details about current events, forgetting events in your own life history, losing awareness of who you are, problems choosing proper clothing, hallucinations, arguments, striking out, violent behavior, delusions, depression, agitation, and difficulty performing basic tasks like preparing meals and driving. At end stages of Alzheimer's Disease, a person can no longer survive without assistance and can no longer understand language, recognize family members, perform basic activities of daily living such as eating, dressing, and bathing.

The term “Parkinson's Disease”, as used herein, refers to a disorder of the brain characterized by shaking (tremor) and difficulty with walking, movement, and coordination. The disease is associated with damage to a part of the brain that is involved with movement. Symptoms of Parkinson's disease may include but are not limited to, muscle rigidity, stiffness, difficulty bending arms or legs, poor posture, loss of balance gait changes, difficulty initiating any voluntary movement, small steps followed by the need to run to maintain balance, inability to resume movement, myalgia, shaking, tremors altered facial expression, voice/speech changes, loss of fine motor skills, frequent falls, decline in intellectual function, and a variety of gastrointestinal symptoms.

The term “Cystic Fibrosis”, as used herein, refers to an inherited disease that affects sodium channels in the body and causes respiratory and digestive problems.

Symptoms include, but are not limited to, no meconium (bowel movements) in first 24 to 48 hours of life, stools, pale or clay colored, foul smelling, or stools that float, salty skin, recurrent persistent respiratory infections, coughing/wheezing, weight loss, diarrhea, delayed growth, and easy fatigue.

The term “patient at risk”, as used herein, refers to any person or patient in whom a medical professional considers possible that at least one symptom of neurodegenerative disease may occur. Such subjects may, for example, be from families where other members have had such symptoms but the subject has not shown symptoms.

Additionally, subjects at risk may be individuals in which there is a genetic history of such symptoms. Consequently, a patient at risk may receive an amyloid fibril inhibitor protein as a prophylactic therapeutic.

The term “attach”, “attachment”, or “attaching”, as used herein, refers to any physical relationship between molecules that results in forming a stable complex. The relationship may be mediated by physico-chemical interactions including, but not limited to, ionic attraction. hydrogen bonding, covalent bonding, Van der Waals forces or hydrophobic attraction. Also, a molecule, such as an amyloid fibril inhibitor protein, may be considered attached when fluorescence is detected during a thioflavin T assay.

### GENERAL DESCRIPTION OF THE INVENTION

The invention generally relates to the treatment of patients with Alzheimer's disease, patients at risk for Alzheimer's disease, and other neurodegenerative diseases. Embodiments of the present invention contemplate novel materials and methods for the treatment of such patients. In one embodiment, the present invention contemplates proteins and protein variants that function by decreasing the presence or formation of amyloid fibrils, protofibrils and soluble oligomers that give rise to amyloid plaques.

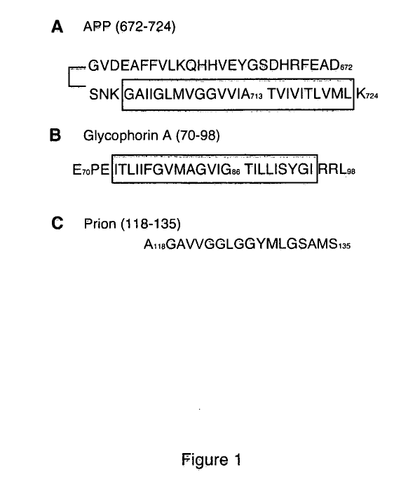

Neuronal damage caused by the toxic aggregation of misfolded proteins is associated with a wide variety of neurodegenerative diseases. Although the present invention is not limited by any particular theory, it is believed that the insoluble plaques characteristic of Alzheimer's disease are formed from amyloid-β (Aβ) peptides with lengths varying from 38-43 amino acids. Glenner et al. “Alzheimer's Disease And Down's Syndrome: Sharing Of A Unique Cerebrovascular Amyloid Fibril Protein” Biochem. Biophys. Res. Commun. 122:1131-1135 (1984); and Glenner et al., “Alzheimer's Disease: Initial Report Of This Purification And Characterization Of A Novel Cerebrovascular Amyloid Protein” Biochem. Biophys. Res. Commun. 120:885-890 (1984). The Aβ₁₋₄₂ peptide (FIG. 1A; SEQ ID NO: 157), the most potent peptide which produces amyloid fibrils, is generated by the action of β-secretase and γ-secretase on the amyloid precursor protein (APP); β-secretase cleaves the extracellular sequence of the protein, while γ-secretase cleaves in the middle of the membrane-spanning sequence. Selkoe et al., “Translating Cell Biology Into Therapeutic Advances In Alzheimer's Disease” Nature 399:A23-A31 (1999); and De Strooper et al., “Alzheimer's Disease—An Inflammatory Drug Prospect” Nature 414:159-160 (2001).

Aβ₁₋₄₂ (SEQ ID NO: 157) undergoes two structural transformations. The first transformation changes the protein from a membrane-spanning α-helix to a soluble peptide fibril with an extended β-strand structure. The second transformation changes the protein from an extended β-strand structure to a cross β-sheet structure that plays a significant role in the progression of Alzheimer's disease. Although it is not necessary to understand the mechanism of an invention, it is believed that glycine, which is highly prevalent in Aβ₁₋₄₂ (SEQ ID NO: 157), potentiates these structural changes.

First, glycine is known to function as a helix breaker in aqueous environments and is often present in proteins that convert from an α-helix to a β-sheet. Dalal et al, “Transforming α-Helices And β-Sheets” Folding & Design 2:R71-R79 (1997). Second, glycine has a higher preference for β-sandwich folds than α-sheet alone; a property that stabilizes β-sheet-to-p-sheet packing. Glycine, in the context of hydrophobic and β-branched amino acids, aids correct membrane peptide insertion.

Current Alzheimer's disease treatments include tacrine hydrochloride (Cognex®), donepezil hydrochloride (Aricept®), rivastigmine hydrochloride (Excelon®) and galantamine hydrochloride (Reminyl®) all of which presumably act by elevating acetylcholine concentrations in the cerebral cortex by inhibiting an enzyme that degrades acetylcholine (i.e., for example, acetylcholinesterase). Other drugs under investigation, may counteract the destructive or toxic effects of a “glutamate cascade”. Glutamate is believed release in excessive amounts during brain cell death as a consequence of stroke, Alzheimer's disease, or Parkinson's disease. None of these neurodegenerative disorder treatments address the formation of an amyloid peptide fibril formation.

### DETAILED DESCRIPTION OF THE PREFERRED EMBODIMENTS

The present invention generally relates to novel materials and methods for the treatment of patients with, or at risk for, Alzheimer's disease or other neurodegenerative diseases. As noted above, presently available Alzheimer's treatments increase brain acetylcholine levels. However, none of these methods has proved curative or shown to slow Alzheimer's physiological progression. In fact, the currently available treatments only lessen disease symptoms and have little, if any, effect on disease progression. The present invention contemplates novel pharmaceutical compositions and related methods to improve treatment for Alzheimer's patients and patients at risk for Alzheimer's disease.

Drugs are ineffective for the treatment of causative agents of Alzheimer's disease. The current collection of drugs used to treat Alzheimer's disease do no more than temporarily relieve symptoms of the disease. It has been long recognized in the art that new compounds are needed to address the causative agents of this disease. Although the present invention is not limited to any particular theory, it is believed that amyloid fibril plaque formation in brain tissue contributes to Alzheimer's disease etiology. In this regard, in some embodiments of the present invention, proteins or protein mimetics are contemplated that prevent the formation (i.e., for example, by polymerization) of amyloid fibrils or breakdown (i.e., for example, by depolymerization) amyloid fibrils that have already formed. Currently, there are no protein-based therapeutics for the treatment of subjects with symptoms of Alzheimer's disease or other neurodegenerative diseases.

1. Compositions of the Present Invention

In one embodiment, an amyloid fibril inhibitor protein composition comprises a peptide including, but not limited to, SEQ ID NOs: 1-4. While it is not necessary to understand the mechanism of an invention, it is believed that an amyloid peptide (i.e., for example, SEQ ID NO: 157) dissolves (i.e., for example, by depolymerization) when exposed to the protein inhibitor (i.e., for example, SEQ ID NOs: 1-4). It is further believed, that the dissolution process results from a competitive inhibition process. Further, it is believed that the competitive inhibition results from a protein inhibitor association with Aβ₁₋₄₂ peptides. Consequently, this association disrupts the soluble oligomers that lead to the growth and deposition of fibrils in affected brain cells.

Although it is not necessary to understand the mechanism of an invention, it is believed that amyloid fibril inhibitor proteins bind to amyloid fibrils (e.g., those associated with Alzheimer's disease). In one embodiment, amyloid fibril inhibitor protein compositions comprise three functional portions; two protein binding portions (i.e., Xaa1 and Xaa2) and a polar portion (i.e., Xaa3). In one embodiment, a protein binding portion binds to an amyloid fibril β-pleated sheet or soluble oligomer (i.e., a target peptide), and a polar portion increases the solubility of the inhibitor-amyloid fibril complex.

Although the present invention is not limited to any particular mechanism, it is believed that amyloid peptide fibrils form plaques comprising β-pleated sheets by binding to each other (i.e., for example, during Alzheimer amyloid peptide fibril plaque formation). It is further believed that the amyloid fibril inhibitor proteins of the present invention disrupt these amyloid peptide fibril plaque β-pleated sheets by preventing amyloid fibril polymerization (i.e., for example, by competitive interactions). In some embodiments, the amyloid fibril inhibitor proteins of the present invention comprise both protein binding portions and a polar portion. In one embodiment, polar portions alternate with protein binding portions.

The amyloid fibril inhibitor proteins of the present invention may be synthesized by methods known in the art, for example: Deming et al., “Methods And Compositions For Controlled Polypeptide Synthesis” U.S. Pat. No. 6,632,922 (2003); Ray et al., “Method Of Cyanide Salt Production” U.S. Pat. No. 6,649,136 (2003); Tam J.P., “Method For Synthesis Of Proteins” U.S. Pat. No. 6,310,180 (2001); and

Elmore D. T., “Solid Phase Peptide Synthesis” U.S. Pat. No. 4,749,742 (1988) (all herein incorporated by reference). Peptides may also be synthesized on automated peptide synthesizing machines such as the Symphony/Multiplex™ automated peptide synthesizer (Protein Technologies, Inc, Tucson, Ariz.) or Perkin-Elmer Model 433A automated peptide synthesizer (Applied Biosystems, Foster City, Calif.).

As is known in the art, peptides can be synthesized by linking an amino group to a carboxyl group that has been activated by reaction with a coupling agent, such as dicyclohexylcarbodiimide (DCC). The attack of a free amino group on the activated carboxyl leads to the formation of a peptide bond and the release of dicyclohexylurea.

Protection of other potentially reactive groups (i.e., for example, side chain amino and carboxyl groups) is frequent. For example, the amino group of the component containing the activated carboxyl group can be blocked with a tertbutyloxycarbonyl group. This protecting group can be subsequently removed by exposing the peptide to dilute acid, which leaves peptide bonds intact. With this method, peptides can be readily synthesized by a solid phase method by adding amino acids stepwise to a growing peptide chain that is linked to an insoluble matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

In one embodiment, the present invention contemplates synthetic protein mimetics comprising peptides having sequence homology to RGTFQGKF (SEQ ID NO: 1) or any variants thereof, for example, as shown in Table 1 and Table 2.

One common methodology for evaluating sequence homology is the Monte Carlo analysis. According to this analysis, a Z value is obtained by an algorithm whereby a Z value greater than 6 indicates probable significance, and a Z value greater than 10 is considered to be statistically significant. Pearson et al., Proc. Natl. Acad. Sci. (USA), 85:2444-2448 (1988); and Lipman et al., Science, 227:1435-1441 (1985). In the present invention, synthetic polypeptides useful in the treatment of patients with Alzheimer's disease or patients at risk for Alzheimer's disease are those peptides having probable statistically significant sequence homology and similarity (i.e., a Monte Carlo Z value exceeding 6).

One method of peptide synthesis involves the use of protecting groups (i.e., for example, fluorenylmethyloxycarbonyl; Fmoc). Fmoc chemistry, as it applies to solid phase peptide synthesis, is well known in the art. All Fmoc groups that protect side chain functional groups of individual amino acids are acid labile, while the N-terminal amino function of the amino acid is protected by Fmoc groups which are base labile. Therefore, incorporation of new amino acids to a growing peptide involves treating an Fmoc-derivatized amino acid that is already attached to a resin with a base (e.g., for example, 20% piperidine/DMF) and adding a new Fmoc amino acid activated ester along with an appropriate activator (e.g., N-hydroxy benzotriazole; HOBT). Attaching the new amino acid to the peptide chain involves coupling agents including, but not limited to, i) 1-H-Benzotriazolium (HBTU)/HOBT; ii) (N-[Dimethylamino)-1H-1,2,3,-Triazolo [4,5-b] Pyridin-1-YLMethylene]-N-Methylmethanaminium Hexafluophosphate N-Oxide (HATU)/1-Hydroxy-7- Azabenzotriazole (HOAT); iii) (Benzotriazol-1-yl-10 oxytripyrrolidinophosphonium hexafluorophosphate (PyBOP)/HOBT; or iv) OPFP-preactivated amino acids/HOBT. Peptides can be synthesized as the free carboxyl or as the C-terminal amide. The N-terminus can be free or acetylated. In addition, the bromoacetylation of the N-terminus and inclusion of norleucine for quantitating the peptide loading onto the carrier protein are routine procedures. Incorporation of unusual amino acid derivatives may be accomplished by use of appropriate Fmoc activated esters. Both the Symphony and the Perkin Elmer/ABI instruments incorporate the use of HBTU/HOBT for its coupling chemistry. Consequently, each protected amino acid is coupled as the free acid. The first residue may be attached to a HMP (Wang) resin (p-benzyloxybenzyl alcohol resin) using DMAP (4-(Dimethylamino)pyridine) following by a capping step using benzoic anhydride or one can use a variety of preloaded resins.

Synthetic peptides may be analyzed by reverse phase HPLC and mass spectrometry. HPLC conditions for one analysis are as follows: i) load sample onto a 0.4×25 cm Vydac C18 analytical column; ii) apply 10-50% Acetonitrile (0.1% TFA)/water for a 40 minute linear gradient at a flow rate of 1.0 ml/min; iii) detect effluent using a UV detector at 220 nm (i.e., for example, a Beckman Diode Array Detector Model 168); and iv) record detector signals at a chart speed of 0.5 cm/min. For peptides that contain one or more cysteine residues, it is not uncommon to obtain multiple peaks on an HPLC chromatogram due to oxidation of the sulflhydryls. Likewise, methionine can be oxidized to its corresponding sulfoxide which will exhibit itself as an additional peak on the HPLC chromatogram. Other peaks can be produced by incomplete deprotection of Arg, deletion peptides (no capping) or truncated peptides (with capping). Unpurified preparations normally contain 60-95% of the expected product. However, this tends to be peptide specific. A typical HPLC preparative run can handle 100-150 mg of crude peptide. An exemplary preparative HPLC column comprises a Millipore 25 mm×10 cm C₁₈ column RCM. Separation conditions may vary and are dependent on the analytical HPLC chromatogram profile which may require either isocratic or gradient elutions.

Mass spectrometric analysis is also an excellent technique for structural analysis. For example, one such instrument is the Voyager-DE RP Biospectrometry Workstation (AME Bioscience, Norway). This spectrometer is a matrix-assisted laser desorption ionization-time of flight instrument (MALDI-TOF). It is equipped with delayed extraction (DE) for improved mass accuracy and a reflector (RP) for fragmentation analysis. If the N-terminal amino function of the peptide is free, then N-terminal sequencing of the peptide is possible.

Under normal synthetic conditions, where the protein of interest constitutes the majority of the preparation, yields are of the order of 50%. However, this is highly dependent upon the complexity of the amount of impurities found in the crude peptide as well as of the purification conditions themselves.

2. Glycine Plays A Role In Neurodegenerative Protein Formation

Glycine has different roles in the structure and function of soluble and membrane proteins. A soluble protein comprising glycine has a high propensity for reverse turns, particularly in the proximity of polar amino acids, such as, Asn, Asp, Ser and Glu.

Wilmot et al., “Analysis And Prediction Of The Different Types Of Beta-Turn Proteins” Folding & Design 2:R71-R79 (1997). Further, glycine is known to facilitate helix association by acting as a “molecular notch”. Javadpour et al., “Helix Packing In Polytopic Membrane Proteins: Role Of Glycine In Transmembrane Helix Association” Biophys. J 77:1609-1618 (1999). A transmembrane protein usually has a high occurrence of glycine in the hydrophobic membrane spanning helix. Eilers et aL, “Comparison Of Helix Interactions In Membrane And Soluble Alpha-Bundle Proteins” Biophys. J. 82:2720-2736 (2002). Glycine is thought to affect peptide solubility since, in some peptides, when glycine is substituted with alanine an insoluble fibril forms. Protein fibril formation is commonly associated with Alzheimer disease, but is suspected as a neurodegenerative process in other diseases.

Protein fibrillization occurs in vivo as a result of improper folding or misfolding. Diverse diseases arise from protein misfolding and are now grouped under the term “protein conformational diseases”, including but not limited to, most of the neurodegenerative disorders such as Alzheimer's disease, Parkinson's disease, the prion encephalopathies and Huntington's disease, as well as cystic fibrosis, sickle cell anemia and other less common conditions. The hallmark event in these diseases is a change in the secondary and/or tertiary structure of a normal, finctional protein, leading to the formation of protein fibrils with various supramolecular organizations. In most cases, the these structurally well-defined fibrils form amyloid deposits. These conformational changes may promote the disease either by increased toxicity or decreased biological function of the natively folded protein. Merlini et al., “Protein Aggregation” Clin Chem Lab Med. 39:1065-75 (2001).

Glycines are also abundant in non Alzheimer-associated fibril proteins, such as α-synuclein, the protein associated with Parkinson's disease. α-Synuclein has an α-helical secondary structure that converts to β-sheet upon fibril formation. Kessler et al., “The N-Terminal Repeat Domain Of α-Synuclein Inhibits β-Sheet And Amyloid Fibril Formation” Biochemistry 42:672-678 (2003). α-Synuclein's highly fibrogenic core (i.e., residues 60-85) contains several glycines in the context of a long stretch of hydrophobic residues similar to the C-terminus of Aβ₁₋₄₂ (SEQ ID NO: 157).

Glycine is also prevalent in the prion protein, a misfolded protein that forms insoluble plaques. Prion fibrils are believed responsible for bovine spongiform encephalopathy (i.e., Chronic Wasting Disease, otherwise known as “mad cow disease”).

Finally, glycines and valines are present in the lung surfactant protein SP-C, which contains a long hydrophobic α-helix whose membrane association is controlled in part by two palmitoyl groups. Johansson, J., “Membrane Properties And Amyloid Fibril Formation Of Lung Surfactant Protein” Biochem. Soc. Trans. 29:601-606 (2001).

Removal of the palmitoyl groups is associated with fibril formation. Gustafsson et al., “Amyloid Fibril Formation By Pulmonary Surfactant Protein” FEBS Lett. 464:138-142 (1999). Fibril peptides comprising lung surfactant protein is believed responsible for some symptoms of cystic fibrosis.

Previous studies showed that glycine might mediate helix-helix interactions in membrane proteins. Javadpour et al., “Helix Packing In Polytopic Membrane Proteins: Role Of Glycine In Transmembrane Helix Association” Biophys. J. 77:1609-1618 (1999). The data presented above reveal a surprising structural versatility for glycine which appears to be dependent on the nature of the protein sequence and its environment.

One embodiment of the present invention contemplates that a dovetail packing design for amyloid plaques effects an inhibitor's efficacy to disrupt fibril formation. In another embodiment, the association of amyloid β-sheets to form a protofibril is facilitated by surface groves created by alternating glycine residues which allow the large hydrophobic side chains to pack into a dovetail fashion. It is not known whether the β-sheets in the protofibril pack in a parallel of antiparallel fashion.

The occurrence of amino acids in β-sheet secondary structure for the 20 representative (unique) proteins comprising the β-sandwich family defined by the CATH database (Architecture 2.60) has been reported in the art. Orengo et al., “CATH—A Hierarchic Classification Of Protein Domain Structures” Structure 5:1093-1108 (1997). The amino acid preferences in the p-sandwich fold were calculated as the percentage of each amino acid having β-structure in the β-sandwich fold. The amino acid preferences for β-sheet secondary structure were obtained from the literature. Wilmot et al., “Analysis And Prediction Of The Different Types Of Beta-Turn Proteins” J. Mol. Biol. 203:221-232 (1998); and Williams et al., “Secondary Structure Predictions And Medium Range Interactions” Biochem. Biophys. Acta 916:200-204 1987).

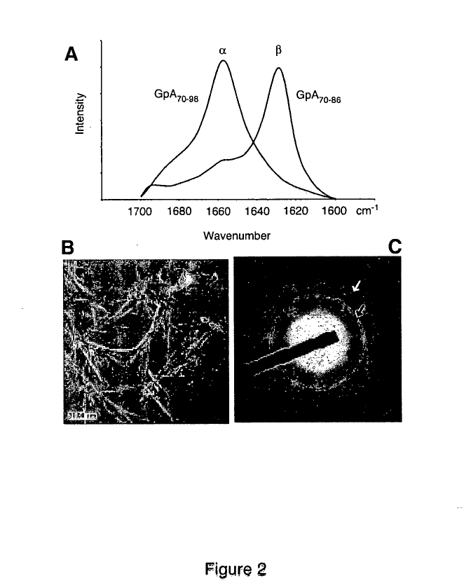

The glycophorin A transmembrane sequence contains four glycine residues and is known to form a well-defined α-helix in membrane bilayers. (See FIG. 1B). Smith et al., “Determination Of Helix-Helix Interactions In Membranes By Rotational Resonance NMR” Proc. Natl. Acad. Sci., USA 92:488-491 (1995). FIG. 2A presents an exemplary Fourier transform infrared spectroscopy (FTIR) spectra of glycophorin A peptides following dimyristoylphosphatidylcholine (DMPC) reconstitution into membrane bilayers.

FTIR spectra were obtained on a Bruker IS 66V/S spectrometer. Peptides were first co-solubilized with octyl-β-glucoside and DMPC and then the detergent was removed by dialysis. The resulting multilamellar vesicle dispersions at a concentration of mg lipid/mL were spread on a germanium internal reflection element and bulk water was removed using a slow flow of N₂ gas to form a multilamellar lipid-peptide film. The full length transmembrane sequence, GpA₇₀₋₉₈, forms a membrane spanning α-helix characterized by an amide I vibration at 1657 cm⁻¹. In striking contrast, the truncated GpA₇₀₋₈₆ peptide, which is no longer able to span the bilayer, adopts almost exclusively β-structure (i.e., the isolated transmembrane domain peptide fragment). This truncated GpA₇₀₋₈₆ forms fibrils similar to the Aβ₁₋₄₂ peptide. (See FIGS. 2B and 2C). The full length transmembrane peptide does not form fibrils.

Before this invention was conceived, it was not known whether glycine had a structural role in the formation of amyloid fibrils from the Ap-peptides. It was known, however, that amyloid fibrils have a cross p-sheet structure where β-strands bond along the direction of the polymerized fibril (i.e., the protofibril axis). Sunde et al., “From The Globular To The Fibrous State: Protein Structure And Structural Conversion In Amyloid Formation” Q. Rev. Biophys 31:1-39 (1998). However, the mechanism for how the β-sheets associate in the direction perpendicular to the protofibril axis was not known and has relevance in understanding how amyloid fibrils form. There was speculation in the art that since there was very low sequence identity between different fibril forming peptides, amyloid fibrils might be stabilized by main chain interactions. Chiti, et al. “Designing Conditions For In Vitro Formation Of Amyloid Protofilaments And Fibrils” Proc. Natl. Acad. Sci, USA 96:3590-3594 (1999). In one embodiment, this invention contemplates that glycine may mediate, at least in part, the association of amyloid β-sheets.

High resolution techniques provide an opportunity to address whether glycine provides a complementary surface for amyloid β-sheet packing. The resolution necessary to determine the detailed fibril structures is not possible using x-ray crystallography or by high-resolution solution NMR because the samples are inherently insoluble. Several groups, however, have obtained high-resolution data using solid-state NMR. Castellani et al., “Structure Of A Protein Determined By Solid-State Magic-Angle-Spinning NMR Spectroscopy” Nature 420:98-102 (2002); and Burkoth et al., “Structure Of The β-Amyloid (10-35) Fibril” J. Am. Chem. Soc. 122:7883-7889 (2000).

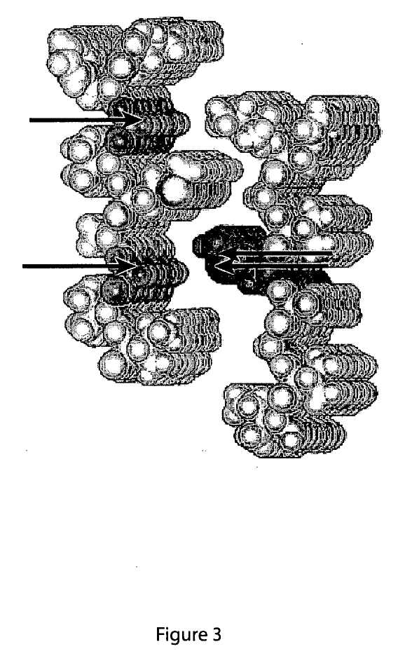

A single high resolution contact point is suspected to tightly constrain the β-sheet-to-β-sheet packing arrangement since the intra-sheet structure is known to be in-register and parallel. Tycko, R., “Insights Into The Amyloid Folding Problem From Solid-State NMR” Biochemistry 42:3151-3159 (2003). β-Sandwich modeling formed from the C-terminal peptide in the arrangement shown in FIG. 3 predicts an inter-β-sheet distance of 4-6 Å between the α-¹³C of Gly³⁷ and ¹³CH₃ of Met³⁵. The intra-β-sheet distance between these amino acids is predicted to be 8-10 Å, outside of the detection range of the NMR experiment.

This previous solid state NMR data focuses on defining the Aβ-peptide secondary structure and how the amyloid β-strands are oriented relative to one another within β-sheet. Using solid-state NMR, detailed structural models for Aβ₁₋₄₀ protofibrils have been proposed. Petkova et al., “A Structural Model For Alzheimer's β-Amyloid Fibrils Based On Experimental Constraints From Solid State NMR” Proc. Natl. Acad. Sci, USA 99:16742-16747 (2002). An integrated solid state NMR/electron microscopy study shows that the amyloid protofibril is the narrowest component within an amyloid fibril and has a cross section consistent with two Aβ-peptides having a large bend near Gly¹². Antzutkin et al., “Supermolecular Structural Constraints On Alzheimer's β-Amyloid Fibrils From Electron Microscopy And Solid-State Nuclear Magnetic Resonance” Biochemistry 41:15436-15450(2002).

Amyloid fibril NMR studies have identified a hydrophilic, unstructured, N-terminus comprising 10 amino acids and a hydrophobic, glycine-rich, C-terminus having an extended β-structure which hydrogen bonds in a parallel arrangement within a β-sheet. Antzutkin et al., “Supermolecular Structural Constraints On Alzheimer's β-Amyloid Fibrils From Electron Microscopy And Solid-State Nuclear Magnetic Resonance” Biochemistry 41:15436-15450 (2002); and Balback et al., “Supramolecular Structure In Full-Length Alzheimer's β-Amyloid Fibrils: Evidence For A Parallel β-Sheet Organization From Solid-State Nuclear Magnetic Resonance” Biophys. J. 83-1205-1216 (2002). Clearly, amyloid peptide fibrils have a C-terminus that is considerably more hydrophobic than the N-terminus and is thought to be the protofibril's core. These previous studies leave unanswered the question as to how the β-sheet formed by the C-terminus associates to form protofibrils.

FIG. 3 presents a possible packing arrangement of the C-terminal 15 amino acids of Aβ₁₋₄₂ as contemplated in one embodiment of the present invention. This hypothesis is supported by observations that alternating large and small side chains are known to occur in-register within an amyloid β-sheet. Antzutkin et al., “Multiple Quantum Solid-State NMR Indicates A Parallel, Not Antiparallel, Organization Of β-Sheets In Alzheimer's β-Amyloid Fibrils” Proc. Natl. Acad. Sci., USA 97:13045-13050 (2000).

3. β-Amyloid Plaque-Dissolving Agents and Antagonists

A. Designing Peptide Variants And Non-Peptide Mimetics

Compounds mimicking the necessary conformation of the sequences of the present invention for recognition and binding to Aβ₁₋₄₂ are contemplated as within the scope of this invention. For example, in one embodiment, mimetics of the RGTFQGKF (SEQ ID NO: 1) protein and variants thereof, and all other proteins within the scope of the present invention are contemplated. A variety of designs for such mimetics are possible and multiple methods are known in the art for creating such compounds. Lobl et al., “Cyclic Cell Adhesion Modulation Compounds” U.S. Pat. No. 5,192,746 (1993); Burke, Jr. et al, “Analogs Of Viscosin And Their Uses” U.S. Pat. No. 5,169,862 (1992); Bischoff et al., “Bcl-2 and R-Ras Complex” U.S. Pat. No. 5,539,085 (1996); Aversa et al., “Antibodies To The Slam Protein Expressed On Activated T Cells” U.S. Pat. No. 5,576,423 (1996); Shashoua V. E., “GABA Esters And GABA Analog Esters” U.S. Pat. No. 5,051,448 (1991); and Gaeta et al., “Bivalent Sialyl X Saccharides” U.S. Pat. No. 5,559,103 (1996) (all herein incorporated by reference).

Synthesis of non-peptide compounds that mimic peptide sequences is also known in the art. For example, non-peptide antagonists mimicking the Arg-Gly-Asp sequence has been reported. Eldred et al, J. Med. Chem. 37:3882 (1994). Further, the general nature regarding non-peptide mimetic synthesis has been completely reviewed. Ku et al, J. Med. Chem. 38:9 (1995). X-ray crystallography has been reported to provide sufficient information to facilitate non-peptide mimetic compound designs using computer software programs. Beamer et al., “Three-Dimensional Structure Of Bactericidal/Permeability Increasing Protein (BPI)” U.S. Pat. No. 6,093,573 (2000)(herein incorporated by reference). In one embodiment, the present invention contemplates compositions comprising non-peptide mimetics of amyloid peptide fibril plaque inhibitors (i.e., for example, RGTFQGKF (SEQ ID NO: 1).

Non-peptide mimetic compositions contemplated by the present invention may be determined using an NMR structure of an amyloid fibril (i.e., for example, a glycophorin A fibril). An NMR structure useful in designing these new and specific agents (i.e., for example, non-peptide mimetic compositions) include, but are not limited to, organic compounds and may be exemplified by the molecular surface shown in FIG. 3. In one embodiment, a non-peptide mimetic composition comprises a center-to-center spacing of approximately 13.2 angstroms having a depth of approximately 4-6 angstroms, thereby reflecting the surface grooves created by the GxxG motif shown in FIG. 3.

The program DOCK (Kuntz et al. J. Mol. Biol., 161:269-288 (1982)) may be used to analyze an active site or ligand binding site and suggest ligands with complementary steric properties. Several methodologies for searching three-dimensional databases to test pharmacophore hypotheses and select compounds for screening are available. These include the program CAVEAT (Bacon et al. J. Mol. Biol., 225: 849-858 (1992)) which uses databases of cyclic compounds which can act as “spacers” to connect any number of chemical fragments already positioned in the active site. This allows one skilled in the art to quickly generate hundreds of possible ways to connect the fragments already known or suspected to be necessary for tight binding. The program LUDI (Bohm et al. J. Comput.-Aid. Mol. Des., 6:61-78 (1992)) can determine a list of interactions sites into which to place both hydrogen bonding and hydrophobic fragments. LUDI then uses a library of approximately 600 linkers to connect up to four different interaction sites into fragments.

Then smaller “bridging” groups such as —CH2—and —COO— are used to connect these fragments. For example, for the enzyme DHFR, the placements of key functional groups in the well-known inhibitor methotrexate were reproduced by LUDI. Rotstein et al., J. Med. Chem., 36:1700-1710 (1992)).

Rational mimetic design also employs other computer systems capable of forming a representation of a protein's three-dimensional structure, including but not limited to RIBBON (Priestle, J., J. Mol. Graphics 21:572 (1988)), QUANTA (Polygen), INSITE (Biosyn), or NANOVISION (American Chemical Society). The usefulness of such analyses is known by the art. Hol et al. In: Molecular Recognition: Chemical and Biochemical Problems, Roberts, S. M. (ed.); Royal Society of Chemistry; pp. 84-93 (1989)); Hol W. G. J., Arzneim-Forsch. 39:1016-1018 (1989); and Hol W. G. J., Agnew. Chem. Int. Ed. Engl. 25:767-778 (1986).

In accordance with the methods of conventional drug design, a mimetic amyloid inhibitor compound may be obtained by randomly testing molecules whose structures have an attribute in common with the structure of any amyloid fibril inhibitor protein contemplated by the present invention. The quantitative contribution that results from a change in a particular group of a binding molecule can be determined by measuring the capacity of competition or cooperativity between the amyloid fibril inhibitor protein and the putative mimetic.

In one embodiment of rational drug design, a mimetic amyloid inhibitor is designed to share an attribute of the most stable three-dimensional conformation of an amyloid fibril inhibitor protein. Thus, a mimetic analog of an amyloid fibril inhibitor protein may be designed to possess chemical groups that are oriented in a way sufficient to cause ionic, hydrophobic, or van der Waals interactions that are similar to those exhibited by an amyloid fibril inhibitor protein.

In another embodiment of rational design, the capacity of a particular amyloid fibril inhibitor protein to undergo conformational “breathing” is exploited. Such conformational “breathing”—the transient and reversible assumption of a different molecular conformation—is known in the art, and results from temperature, thermodynamic factors, and from the catalytic activity of a molecule. Knowledge of the 3-dimensional structure of an amyloid fibril inhibitor protein facilitates such an evaluation. An evaluation of the natural conformational changes of an amyloid fibril inhibitor protein facilitates the recognition of potential binding sites, potential sites at which hydrogen bonding, ionic bonds or van der Waals bonds might form or might be eliminated due to the breathing of the molecule, etc. Such recognition permits the identification of the additional conformations that an amyloid fibril inhibitor protein could assume, and enables the rational design and production of mimetic analogs that share such conformations.

In yet another embodiment, screening assays may be used to identify non-peptide mimetic compounds. Such an assay will preferably exploit the capacity of an amyloid fibril inhibitor mimetic to affect fibril plaque polymerization and/or depolymerization.

Screening assays are particularly useful for identifying peptide or oligonucleotide fragments or mimetics of amyloid fibril inhibitor proteins, or mimetic analogs.

The present invention also contemplates synthetic variants of amyloid fibril inhibitor proteins comprising multimers of repeating sequences (See, for example, general formulae (i)-(vii)). In one embodiment of the present invention, it is contemplated that the repeated sequences are selected from the sequences of the group comprising SEQ ID NOs: 1-4. (See Table 1).

TABLE 1

Exemplary amyloid peptide inhibitors and variants thereof.
(SEQ ID NO)

| | | | |
|---|---|---|---|
| RGTFEGKF-NH₂ (1) | RFTGEFKG-NH₂ (2) | RFTGEF-NH₂ (3) | RFTAEF-NH₂ (4) |
| RGTFEGKL (5) | RLTGEFKG (40) | RVFTGEF(75) | RVFTAEF(87) |
| RGTFEGKM (6) | RMTGEFKG (41) | RLTGEF (76) | RLTAEF (88) |
| RGTFEGKI (7) | RITGEFKG (42) | RMTGEF (77) | RMTAEF (89) |
| RGTFEGKV (8) | RVTGEFKG (43) | RITGEF(78) | RITAEF(90) |
| RATFEGKF (9) | RFTAEFKG (44) | RWTGEF (79) | RWTAEF (91) |
| RATFEGKL (10) | RFTAELKG (45) | RYTGEF(80) | RYTAEF(92) |
| RATFEGKM (11) | RFTAEMKG (46) | RFTGEV(81) | RFTAEV(93) |
| RATFEGKI (12) | RFTAEIKG (47) | RFTGEL (82) | RFTAEL (94) |
| RATFEGKV (13) | RFTAEVKG (48) | RFTGEM(83) | RFTAEM(95) |
| RGTFEAKF (14) | RFTGEFKA (49) | RFTGEI (84) | RFTAEI (96) |
| RGTFEAKL (15) | RLTGEFKA (50) | RFTGEW (85) | RFTAEW (97) |
| RGTFEAKM (16) | RMTGEFKA (51) | RFTGEY(86) | RFTAEY(98) |
| RGTFEAKI (17) | RITGEFKA (52) | | |
| RGTFEAKV (18) | RVTGEFKA (53) | | |
| RATFEAKF (19) | RFTAEFKA (54) | | |
| RATFEAKL (20) | RFTAELKA (55) | | |
| RATFEAKM (21) | RFTAEMKA (56) | | |
| RATFEAKI (22) | RFTAEIKA (57) | | |
| RATFEAKV (23) | RFTAEVKA (58) | | |
| RATLEAKF (24) | RLTAEFKA (59) | | |
| RATMEAKF (25) | RMTAEFKA (60) | | |
| RATIEAKF (26) | RITAEFKA (61) | | |
| RATVEAKF (27) | RVTAEFKA (62) | | |
| RGTWEGKW (28) | RWTGEWKG (63) | | |
| RGTYEGKY (29) | RYTGEYKG (64) | | |
| RGTWEGKF (30) | RWTGEFKG (65) | | |
| RGTFEGKW (31) | RFTGEWKG (66) | | |
| RGTYEGKF (32) | RYTGEFKG (67) | | |
| RGTFEGKY (33) | RFTGEYKG (68) | | |
| RATFEAKY (34) | RFTAEYKA (69) | | |
| RATYEAKF (35) | RYTAEFKA (70) | | |
| RATFEAKW (36) | RFTAEWKA (71) | | |
| RATWEAKF (37) | RWTAEFKA (72) | | |
| RATWEAKW (38) | RWTAEWKA (73) | | |
| RATYEAKY (39) | RYTAEYKA (74) | | |

In another embodiment, it is contemplated that the repeated sequences are selected from the core sequences selected from the group comprising the general formulae (i)-(vi) (See Table 2).

TABLE 2

Exemplary amyloid peptide inhibitor core sequence variants.
(SEQ ID NO).

| | | |
|---|---|---|
| Formula (i) | Formula (ii) | Formula (iii) |
| Xaa3-Xaa2-Xaa1 | Xaa3-Xaa2-Xaa3-Xaa1-Xaa3-Xaa1 | Xaa3-Xaa2-Xaa3-Xaa2-Xaa3-Xaa1 |
| (147) | (148) | (149) |

| | | |
|---|---|---|
| F-Xaa2-G (99) | Xaa3-F-Xaa3-G-Xaa3-G (107) | Xaa3-F-Xaa3-F-Xaa3-G (115) |
| F-Xaa2-A (100) | Xaa3-F-Xaa3-G-Xaa3-A (108) | Xaa3-F-Xaa3-F-Xaa3-A (116) |
| F-Xaa2-S (101) | Xaa3-F-Xaa3-G-Xaa3-S (109) | Xaa3-F-Xaa3-F-Xaa3-S (117) |
| W-Xaa2-G (102) | Xaa3-F-Xaa3-A-Xaa3-A(110) | Xaa3-Y-Xaa3-F-Xaa3-G (118) |
| Y-Xaa2-G (103) | Xaa3-F-Xaa3-A-Xaa3-G (111) | Xaa3-W-Xaa3-F-Xaa3-G (119) |
| W-Xaa2-A (104) | Xaa3-F-Xaa3-S-Xaa3-G (112) | Xaa3-F-Xaa3-W-Xaa3-G (120) |
| Y-Xaa2-A (105) | Xaa3-Y-Xaa3-G-Xaa3-G (113) | Xaa3-F-Xaa3-Y-Xaa3-G (121) |
| F-Xaa2-G (106) | Xaa3-W-Xaa3-G-Xaa3-G (114) | Xaa3-Y-Xaa3-F-Xaa3-A (122) |

TABLE 2(cont'd)
Exemplary amyloid peptide inhibitor core sequence variants.
(SEQ ID NO).

| Formula (iv) | Formula (v) | Formula (vi) |
|---|---|---|
| Xaa3-Xaa1-Xaa3-Xaa2 | Xaa3-Xaa1-Xaa3-Xaa1-Xaa3-Xaa2 | Xaa3-Xaa1-Xaa3-Xaa2-Xaa3-Xaa2 |
| (150) | (151) | (152) |
| Xaa3-G-Xaa3-F (123) | Xaa3-G-Xaa3-G-Xaa3-F (131) | Xaa3-G-Xaa3-F-Xaa3-F (139) |
| Xaa3-A-Xaa3-F (124) | Xaa3-A-Xaa3-GXaa3-F (132) | Xaa3-A-Xaa3-F-Xaa3-F (140) |
| Xaa3-S-Xaa3-F (125) | Xaa3-S-Xaa3-GXaa3-F (133) | Xaa3-S-Xaa3-F-Xaa3-F (141) |
| Xaa3-G-Xaa3-W (126) | Xaa3-A-Xaa3-A-Xaa3-F (134) | Xaa3-G-Xaa3-F-Xaa3-F (142) |
| Xaa3-G-Xaa3-Y (127) | Xaa3-G-Xaa3-A-Xaa3-F (135) | Xaa3-G-Xaa3-F-Xaa3-W (143) |
| Xaa3-A-Xaa3-W (128) | Xaa3-G-Xaa3-5-Xaa3-F (136) | Xaa3-G-Xaa3-W-Xaa3-F (144) |
| Xaa3-A-Xaa3-Y (129) | Xaa3-G-Xaa3-G-Xaa3-Y (137) | Xaa3-G-Xaa3-F-Xaa3-Y (145) |
| Xaa3-S-Xaa3-W (130) | Xaa3-G-Xaa3-G-Xaa3-W (138) | Xaa3-G-Xaa3-F-Xaa3-Y (146) |

In one embodiment, a repeating variant comprises RFTGEFKGTF-NH₂ (SEQ ID NO: 153). In another embodiment, a repeating variant comprises RGTFEGKFTG-NH₂ (SEQ ID NO: 154). Further, the present invention contemplates repeating variant embodiments comprising an amino acid sequence of Xaa3-Xaa2-Xaa3-Xaa1-Xaa3-Xaa2-Xaa3-Xaa1-Xaa3-Xaa2 (SEQ ID NO: 155).

B. Making Peptide Fibril Inhibitors More Resistant to Proteases

In one embodiment, it is contemplated that the proteins of the present invention are made more resistant to degradation by protease. This can be achieved several ways.

In one embodiment, the inhibitor proteins of the present invention may be made cyclic by attaching cytosine residues to the amine and carboxyl termini (i.e., SEQ ID NOs:3 and 4) or by attaching crosslinkers (such techniques are known in the art). In another embodiment, the peptide bond between the amino acids can be replaced with a CH₂ group, or the like. In another embodiment, commercially available protecting groups can be attached to the amino and carboxyl termini. In yet another example, one or more L-amino acids can be replaced with D-amino acids (supra).

In another embodiment, it is contemplated that the amyloid fibril inhibitor proteins of the present invention comprise modified amino acids having at least one non-amino acid moieties (i.e., for example, forming a peptoid). In one embodiment, modified amino acids comprise side chains that are different from the non-modified amino acid.

Glycine, for example, can be replaced with an N-alkylated glycine such as N-isobutylglycine. Kwak, et al., “Triple Helical Stabilities Of Guest-Host Collagen Mimetic Structures,” Bioorg Med Chem. 7:153-60 (1999). Alternatively, one or more amino acids maybe completely replaced with one or more non-amino acid moieties. Pueyo, et al., “A Mimetic Of The RGDF-Peptide (arginine-glycine-aspartic acid-phenylalanine) Blocks Aggregation And Flow-Induced Platelet Deposition On Severely Injured Stenotic Arterial Wall. Effects On Different Animal Models And In Humans” Thromb Res. 81:101 -112 (1996).

4. Administering Therapeutics

One embodiment of the present invention contemplates that an amyloid fibril inhibitor protein (i.e., for example, RGTFQGKF (SEQ ID NO: 1), the peptide variant sequences in Tables 1 and 2, and/or variants contemplated by general formulae (i) - (vii)) can be administered systemically to inhibit amyloid plaque formation in Alzheimer's disease patients or patients at risk for Alzheimer's disease. In one embodiment, the administration is selected from the group comprising intravenous, intrathecal, intraperitoneal, or oral. It is contemplated that the inhibitor proteins of the present invention can be administered alone or in combination with supplemental drugs. In one embodiment, a supplemental drug is useful in the treatment of Alzheimer's disease.

It is not intended that the present invention be limited by the particular nature of the therapeutic preparation. For example, such compositions can be provided together with physiologically tolerable liquid, gel or solid carriers, diluents, adjuvants and excipients.

Such compositions are typically prepared as liquid solutions or suspensions, or in solid forms. Oral formulations usually will include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like.

These compositions take the form of solutions, suspensions, tablets, pills, capsules, sustained release formulations, or powders, and typically contain 0. 1% - 95% of active ingredient, preferably 0.2% -70%.

The compositions are also prepared as injectables, either as liquid solutions or suspensions; solid forms suitable for solution in, or suspension in, liquid prior to injection may also be prepared.

The amyloid fibril inhibitor proteins of the present invention are often mixed with diluents or excipients which are physiological tolerable and compatible. Suitable diluents and excipients are, for example, water, saline, dextrose, glycerol, or the like, and combinations thereof. In addition, the compositions may contain minor amounts of auxiliary substances such as wetting or emulsifying agents, stabilizing or pH buffering agents.

The present invention also relates to the administration of a pharmaceutical or sterile composition, in conjunction with a pharmaceutically acceptable carrier, for any of the therapeutic applications discussed above. Such pharmaceutical compositions may comprise any combination of peptide sequences or mimetics of the present invention, in any combination. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a subject alone or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal.

In addition to the active ingredients, these pharmaceutical compositions may contain pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton Pa.).

For any compound, the therapeutically effective dose can be estimated initially either in cell culture assays or in animal models such as mice, rats, rabbits, dogs, or pigs. An animal model may also be used to determine the concentration range and route of administration. Such information can then be used to determine useful doses and routes for administration in humans. Exemplary amounts for administration are also listed elsewhere in this application.

A therapeutically effective dose refers to that amount of active ingredient which ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. In one embodiment, the present invention contemplates a non-toxic amyloid fibril inhibitor peptide. In one embodiment, the non-toxic peptide comprises SEQ ID NO: 1 (I1). Although it is not necessary to understand the mechanism of an invention, it is believed that neurotoxicity is not present with I1 or I6, but is present with an Aβ₁₋₄₂ peptide. (See Example 5)

Any of the therapeutic compositions described above may be applied to any subject in need of such therapy, including, but not limited to, mammals such as dogs, cats, cows, horses, rabbits, monkeys and, most preferably, humans.

5. Drug Discovery

The amyloid fibril inhibitor proteins of the present invention, in certain 5 embodiments, are contemplated for use in diagnostic assays. For example, in one embodiment, the inhibitor proteins comprise positive controls in screening assays for agents that dissolve Aβ₁₋₄₂ amyloid fibril plaques. In another embodiment, the inhibitor proteins of the present invention comprise a compound for investigating cell death pathways (i.e., for example, apoptosis). For example, the inhibitor proteins of the present invention have been shown to inhibit Aβ₁₋₄₂ peptide-induced cell death (See Example 5).

Comparing cell signal pathway activation in cells treated with the sequences of the present invention versus cells treated with other agents or no agents will aid in the determination of cell signal pathways used in Aβ₁₋₄₂ induced cell death. Such knowledge will be instrumental in determining the molecular basis for Alzheimer's disease. Such knowledge will also be useful in finding other treatments for Alzheimer's and similar diseases.

### EXPERIMENTAL

The following examples serve to illustrate certain preferred embodiments and 2 0 aspects of the present invention and are not to be construed as limiting the scope thereof.

In the experimental disclosure which follows, the following abbreviations apply: eq (equivalents); M (Molar); μM (micromolar); mM (millimolar); N (Normal); mol (moles); mmol (millimoles); μmol (micromoles); nmol (nanomoles); g (grams); mg (milligrams); μg (micrograms); L (liters); ml (milliliters); μl (microliters); cm (centimeters); mm (millimeters); μm (micrometers); nm (nanometers); ° C. (degrees Centigrade); MW (molecular weight); U (units); d(days); A (Angstrom); μL (microliter); sec (seconds); ms (milliseconds); Kev (kiloelectrovolts)

### Example 1

### NMR Spectral Studies Determining Aβ₁₋₄₂ Packing Orientation

This example employs nuclear magnetic resonance (NMR) techniques to demonstrate that glycine promotes the formation of amyloid plaques by facilitating amyloid fibril binding. Although it is not necessary to understand the mechanism of an invention, it is believed that hydrophobic transmembrane peptide domains rich in glycine can spontaneously adopt a i-sheet structure and form fibrils (i.e., for example, amyloid fibrils). As discussed above, the secondary protein structure of a glycine-rich model transmembrane peptide (glycophorin A; GpA) has a sequence similar to Amyloid Protein Polypeptide (APP). One truncated GpA peptide mimics Aβ₁₋₄₂behavior and was used as a model for these studies. This truncated GpA peptide (GpA₇₀₋₈₆) corresponds to the transmembrane domain of glycophorin A (i.e., GpA₇₀₋₉₈); a major protein in erythrocyte membrane.

In the experiments below, magic-angle-spinning (MAS) NMR was performed at 360 and 600 MHz on Bruker Avance NMR spectrometers using 4 mm MAS rotors. The cross polarization contact time was 2 ms, two-pulse phase-modulated (TPPM) decoupling was used during the evolution and mixing periods and the dipolar-assisted rotational-resonance (DARR) mixing time was 1 sec. The proton field strength was set to the n =1 rotational resonance condition during the mixing period. Takegoshi et al., “¹³C-¹H Dipolar-Driven ¹³C-¹³C Recoupling Without ¹³C RF Irradiation In Nuclear Magnetic Resonance Of Rotating Solids” J. Chem. Phys. 118:2325-2341 (2003). DARR gives rise to off-diagonal cross peaks in the 2D NMR spectra if the ¹³C . . . ¹³C contacts are less than 6 A apart.

a. GpA70-86 Fibril β-Sheet Parallel Orientation

Two-dimensional solid-state NMR measurements were performed on GpA₇₀₋₈₆ fibrils containing specifically ¹³C-labeled glycine. For these experiments, fibrils were formed using equimolar amounts of two peptides with ¹³C labels at different positions in the GpA₇₀₋₈₆ sequence. The first peptide was labeled at the α- and carbonyl carbons of Gly⁷⁹ and Gly⁸³, respectively, while the second peptide was labeled at the α- and carbonyl carbons of Gly⁸³ and Gly⁷⁹, respectively. These α- and carbonyl carbons are >14 Å apart within each individual (extended) peptide, but are predicted to be ˜3.5 Å from one another if the peptides have a parallel orientation within a β-sheet.

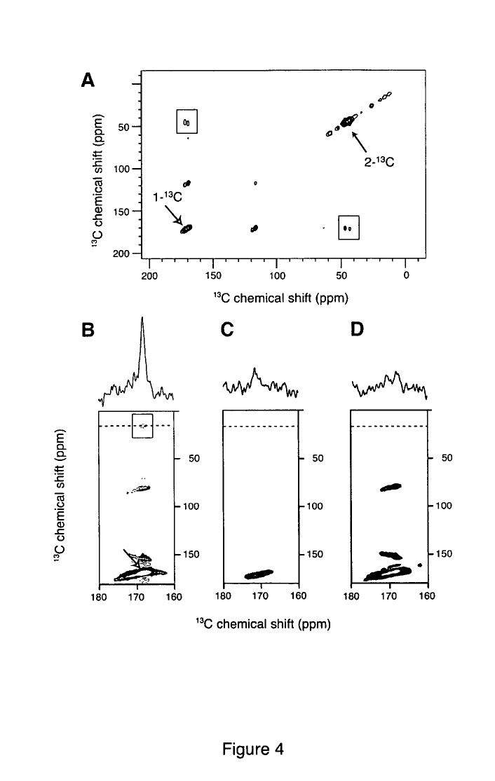

The 2D NMR spectrum analysis used dipolar-assisted rotational-resonance which gives rise to off-diagonal cross peaks if the ¹³C labels are separated by less than ˜5.5 Å. The data demonstrates that both the α-carbon and carbonyl carbon resonances for Gly⁷⁹ and Gly⁸³ are resolved along the diagonal of the 2D NMR spectrum. Cross peak intensity (boxed area) between the α-carbon and carbonyl carbon resonances is consistent with their predicted 3.5 A inter-strand distance indicating that the peptides in the fibrils have a parallel, in-register orientation. (See FIG. 4A). The observation of a parallel, in-register structure for the β-strands supports the conclusion that the alternating large and small side chains produce a series of ridges and grooves running length of the fibril. (See FIG. 3). b. GpA₇₀₋₈₆ Fibril β-Sheet-To-β-Sheet Dovetailing

This experiment tested the idea that the large side chain amino acids on one sheet dovetail into the grooves formed by glycine on an opposing β-sheet using a mixture of two differently labeled pools of GpA₇₀₋₈₆ peptide. A first set of peptides were labeled with a single ¹³C isotope label at the backbone carbonyl carbon of Gly⁷⁹. A second set of peptides were ¹³C -labeled at the Met⁸¹ methyl side chain group. Computer modeling of the GpA₇₀₋₈₆ peptide β-sandwich predicted an inter-β-sheet distance of 3-5 Å between the carbonyl ¹³C of Gly⁷⁹ and the ¹³CH₃ of Met⁸¹. The intra-sheet distance, however, was predicted to be ˜8-10 Å, and outside of the detection range of the NMR experiment used. Solid-state NMR techniques measured the intermolecular distance between Met^{81 13}C-labeled methyl side chains and the carbonyl ¹³C-Gly⁷⁹ on adjacent β-sheets.

An NMR spectrum of an equimolar mixture of ¹³CH₃ Met⁸¹-labeled and 1-¹³C-Gly⁷⁹-labeled GpA₇₀₋₈₆ peptides depicts a well-defined cross peak between Met⁸¹ and Gly⁷⁹ (FIG. 4B). This strong cross peak intensity indicates that the Met⁸¹ side chain is within 5 Å of Gly⁷⁹ on an opposing β-sheet. This data supports the proposal that Met⁸¹ dovetails into the Gly⁷⁹ groove. (See FIG. 3). This conclusion is supported by observations that when fibrils containing only ¹³C-labeled Met81 peptides are measured, this prominent cross peak is absent. (See FIG. 4C).

C. Aβ₁₋₄₂ Peptide Fibril Packing Orientation At The C-Terminal

This experiment was performed to determine whether the packing orientation at the C-terminal of Aβ₁₋₄₂ peptide fibrils association is in a “ridges-into-grooves” or a “dovetail” packing orientation.

A first set of Aβ₁₋₄₂ peptides (SEQ ID NO: 157) was labeled with a single ¹³C isotope at the backbone Gly³⁷ α-carbon. A second set of Aβ₁₋₄₂ peptides was ¹³C -labeled at the Met³⁵ methyl side chain. Solid-state NMR techniques measured the intermolecular distance between ¹³C-CH₃ Met³⁵ side chains on adjacent β-sheets.

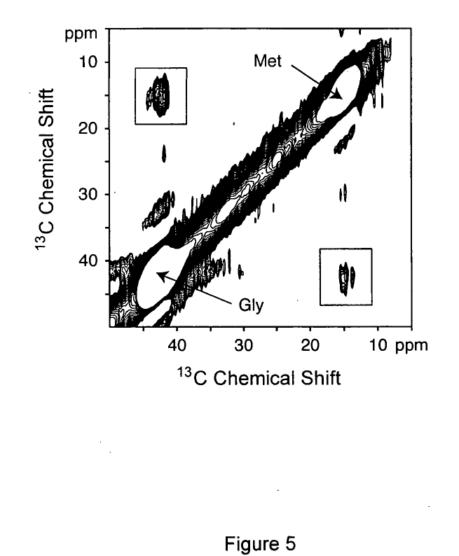

Specifically, a two-dimensional solid-state NMR data spectra of Aβ₁₋₄₂ amyloid fibrils was generated using an: i) equimolar mixture of ¹³CH₃ Met³⁵-labeled peptide and 2-¹³C-Gly³⁷-labelled peptide (FIG. 5). This data was compared to NMR spectra when fibrils were labeled only with ¹³C-CH₃ Met³⁵ (data not shown). The NMR spectra shows a well-defined cross peak between Met³⁵ and Gly³⁷. See FIG. 5. This cross peak between Met³⁵ and Gly³⁷ provides support for the proposed “dovetail packing” of the C-terminal β-sheets. One skilled in the art may recognize that the β-sheets may pack in a parallel fashion (See FIG. 3) or in an antiparallel fashion (not shown).

This cross peak data also supports the proposal that glycines in the C-terminus of Aβ₁₋₄₂ form a packing surface for large bulky side chains present on an opposite β-sheet. This packing configuration is consistent with other studies suggesting that Met³⁵ packs against C-terminal glycines and is involved in the formation of glycyl radicals. Brunelle et al., “The Radical Model Of Alzheimer's Disease: Specific Recognition Of Gly29 And Gly33 By Met35 In A β-Sheet Model Of Aβ: An ONIOM Study” J. Alzheimer's Dis. 4:283-289 (2002).

d. Conclusion

The above data identifies three distinct glycine contributions to the formation of insoluble fibrils (i.e., for example, amyloid plaques).

First, glycine's lack of a side chain increases the flexibility of the polypeptide.

This increased flexibility might facilitate the α-helix to β-sheet transition. This hypothesis is supported by observations that a truncated glycophorin A peptide having glycine-to-alanine substitutions was completely insoluble (data not shown).

Second, glycine can function as a molecular notch which stabilizes β-sheet-to-β-sheet packing. β-Sheet-to-β-sheet packing is a hallmark of the β-sandwich folding motif seen in many soluble proteins where at least two β-sheets stack on top of one another. Comparison of the distributions of amino acids between β-sheets and β-sandwiches show that glycine has a significantly higher percentage of occurrence in the β-sandwich structures (i.e., for example, approximately 5.0%) compared to standard β-sheets (i.e., for example, approximately 2.9%).

Third, β-sheets have an inherent twist due to the chiral structure of all amino acids except glycine. β-sheets containing glycine are flattened due to the lack of side chain chirality. a feature that aids association of two or more β-sheets. Wang et al., “Molecular Simulations Of β-Sheet Twisting” J. Mol. Biol. 262:283-293 (1996).

### Example 2

### SEQ ID NO:1 Prevents Amyloid Peptide Pnlymerizatinn

This example demonstrates the ability of an amyloid fibril inhibitor protein to prevent the polymerization of amyloid peptides into fibrils.

Amyloid fibril formation disruption was hypothesized to occur by an inhibitor protein's specific binding to the C-terminal sequence of Aβ₁₋₄₂ (SEQ ID NO: 157) thereby preventing Met³⁵-Gly³⁷ β-sheet-to-β-sheet contact. An amyloid fibril inhibitor protein having the sequence RGTFEGKF-NH₂ (SEQ ID NO:1) was selected for this experiment. One of skill in the art will realize that this sequence may have a free N-terminus while the C-terminus is protected. One face of an amyloid β-sheet comprises a first repeating amino acid sequence of GXaaFXaaGXaaF (SEQ ID NO: 161) wherein Xaa is any amino acid) and binds to the protein inhibitor's C-terminus with the bulky phenylalanine (i.e., F) side chains packed against Gly³³ and Gly³⁷. The other face of the β-sheet comprises second repeating amino acid sequence of RXaaTXaaEXaaKXaa (SEQ ID NO: 162; wherein Xaa is any amino acid) and is highly polar (i.e., T comprises an —OH; E comprises a —COOH; K comprises an —NH₂) which increases peptide solubility.

Amyloid fibril inhibitor proteins were synthesized on an ABI 430A solid-phase peptide synthesizer (Applied Biosystems, Foster City, Calif.) using tBOC-chemistry.

Cleavage and deprotection was by hydrofluoric acid and purification was by RP-HPLC using linear water-acetonitrile gradients containing 0.1% trifluoroacetic acid. The synthesized peptide amino acid sequences were confirmed by traditional sequence analysis procedures known in the art.

The amyloid fibril inhibitor protein (SEQ ID NO: 1) was incubated at an equimolar ratio with ¹³C-labeled Aβ₁₋₄₂ peptides. During this incubation, electron microscopic images were taken over the course of two weeks in accordance with Example 6. These electron microscopic images revealed no detectable fibrils. In fact, suspensions of Aβ₁₋₄₂ protein (SEQ ID NO: 157) prepared at pH 7 were normally cloudy and formed gels during fibril growth. When the amyloid fibril inhibitor protein (SEQ ID NO: 1) was added, the suspensions became clear in less than 5 seconds and did not form gel compositions; even over a two week period or longer.

A truncated amyloid fibril inhibitor protein (GpA₇₀₋₈₆), that mimics Aβ₁₋₄₂ protein, was incubated with a glycine-rich transmembrane peptide (i.e., similar to APP). This target peptide corresponds to glycophorin A's transmembrane domain comprising a 17 amino acid peptide corresponding to amino acid residues 70-86. Glycophorin A, as a complete protein, is not known to form amyloid-like fibrils. Surprisingly, glycophorin A's transmembrane domain, when observed as a fragment, is unable to span membrane bilayers and adopts almost exclusively β-structure in solution. Consequently, glycophorin A's transmembrane region peptide fragment form fibrils similar to Aβ₁₋₄₂ Similar to amyloid peptides, glycophorin A transmembrane peptide fibril formation can be blocked in the presence of an amyloid plaque inhibitor peptide (SEQ ID NO: 1) at a 1:1 molar ratio.

This assay system has identified several eight amino acid peptides tested as being effective for their ability to inhibit amyloid fibril formation. (SEQ ID NOs: 1-4) The two most effective inhibitors were:

| | |
|---|---|
| RGTFEGKF-NH₂ | SEQ ID NO.: 1 |
| RATFEAKF-NH₂ | SEQ ID NO.: 19 |

### Example 3

### Inhihition of Amyloid Fibral Polymerizatinn

This example presents data showing that SEQ ID NO: 1 inhibits amyloid fibril polymerization in a time and dose-dependent manner.

Amyloid fibrillization was measured by the fluorescence thioflavin T assay. Levine III, H, Methods in Enzymology 309: 274-305 (1999). The Aβ₁₋₄₂ peptide was dissolved and fibrillization was followed over the course of two weeks by monitoring thioflavin T fluorescence. Hou et al., J. Am. Chem. Soc. in press (2004).

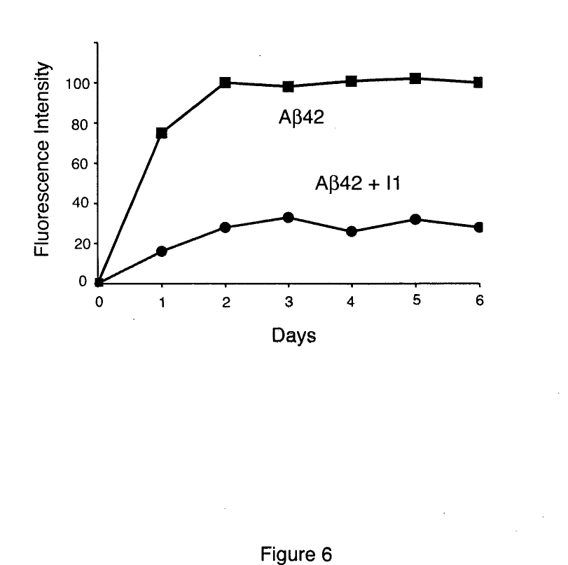

FIG. 6 shows the effect of SEQ ID NO: 1 (I1) when incubated with Aβ₁₋₄₂ peptides at molar ratio of 1:20 over a six (6) day period. The effect of the inhibitor plateaus after three days and shown to be 70%±5 % effective in preventing fibrillization.

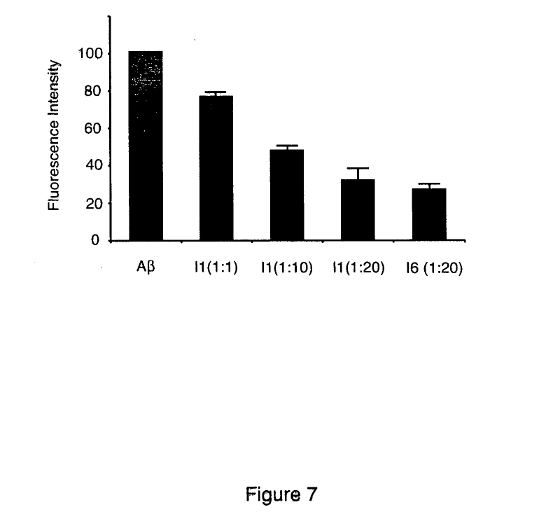

Inhibition activities were also measured by fluorescence intensity over time at the following molar ratios of Aβ₁₋₄₂ peptide to SEQ ID NO:1 (I1) 1:0; 1:1; 1:5; and 1:20. (FIG. 7). The inhibition was compared with a protein fragment NH₂-LPFFD-NH₂ (SEQ ID NO: 160; I6) that has been reported as an effective inhibitor. Soto, et al., Nature Medicine 4:822-826 (1998); and Soto et al., Biochem. Biophys. Res. Comm. 226:672-680 (1996).

### Example 4

### Depolymerizatinn of Preformed Amyloid Fibrils

This example presents exemplary data demonstrating that SEQ ID NO: 1 (I1) depolymerizes pre-existing amyloid fibrils.

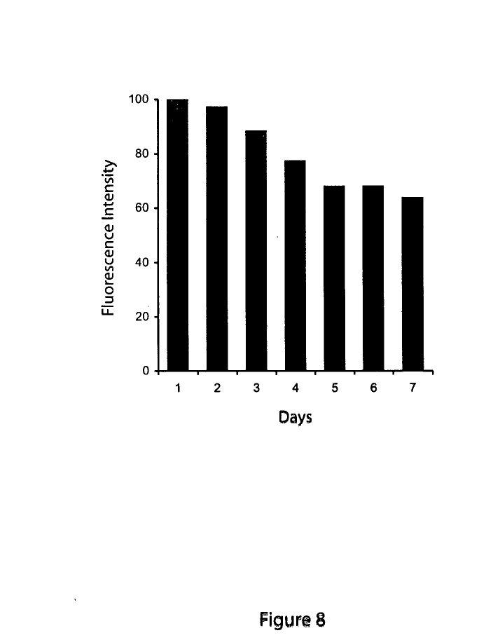

Preformed amyloid fibrils comprising Aβ₁₋₄₂ peptides were incubated with and without SEQ ID NO: 1 (I1) a 1:20 molar ratio. The data clearly show that during the first four (4) incubation days, an apparent linear depolymerization rate occurs. (See FIG. 8) After Day 4, the depolymerization rate and polymerization rate apparently reaches equilibrium.

### Example 5

### Amyloid Fibril Neurotoxicity

This example presents exemplary data demonstrating that the neurotoxic effects of both amyloid fibrils and Aβ₁₋₄₂ peptides may be protected by amyloid fibril peptide inhibitors.

These peptides were tested in cell toxicity assays for their ability to prevent cell death (i.e., for example, by apoptotic mechanisms) induced by the Aβ₁₋₄₂ peptide (SEQ ID NO:157). Furthermore, these tests show that two peptides (i.e., SEQ ID NOs: 1, 19) retain amyloid plaque inhibition activity with either the N- or C-terminal protected or if both the N- and C-terminal protected (data not shown).

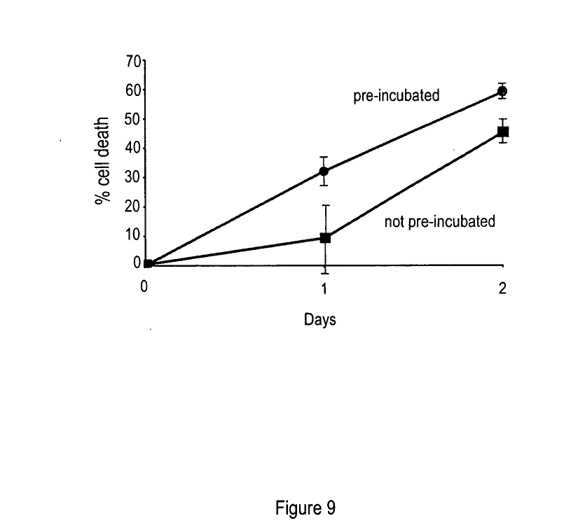

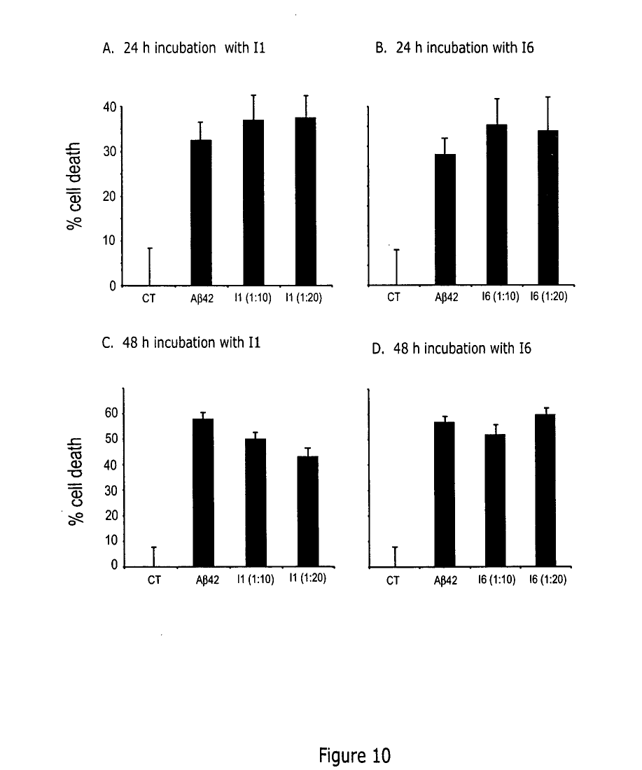

An in vitro neuronal cell culture was grown to confluence and studied by cell count as a measure of neurotoxicity. Neurotoxicity was observed following one or two days of incubation with either polymerized Aβ₁₋₄₂ peptide into amyloid fibrils (ag) or free (i.e., non-polymerized) Aβ₁₋₄₂ peptides (sol). (See FIG. 9). In contrast, however, two contemplated amyloid fibril peptide inhibitors (i.e., SEQ ID NO: 1 (I1) and SEQ ID NO:160 (I6)) were not toxic by themselves when incubated with the neuronal cell culture. See Table 3.

TABLE 3
I1 And I6 Neurotoxicity

| 48 Hour | | | | |
|---|---|---|---|---|
| Incubation | I1 10X | I1 20X | I6 10X | I6 20X |
| % Survival | 91.7 ± 5.6 | 90.5 ± 6.4 | 97.9 ± 4.2 | 97.8 ± 6.6 |

Neither SEQ ID NO: 1 or SEQ ID NO: 160 reduced neurotoxicity when added to the cell culture either before Aβ₋₄₂ peptide were allowed to polymerize or after Aβ₁₋₄₂ peptides were allowed only 24 hours of polymerization. (See FIG. 11A). SEQ ID NO: 1, but not SEQ ID NO: 160, dose-dependently inhibited neurotoxicity when Aβ₁₋₄₂ peptides were allowed 48 hours of polymerization. (See FIG. 11B).

### Example 6

### Electronmicroscopic Analysis of Amyloid Fibril Formation

This example demonstrates the time course of amyloid fibril formation from soluble peptides.

Fibrillized amyloid samples were prepared by incubation of aqueous solutions at an Aβ₁₋₄₂ peptide concentration of approximately 0.2 mM in 1.0 mM phosphate buffer at pH 7.0 with the pH adjusted as necessary by dropwise addition of dilute NaOH or acetic acid. 0.01% NaN₃ was added to inhibit bacterial and fungal growth. The solution was incubated for approximately 10 days at a temperature of 24° C., with gentle rocking (200 rpm). Aliquots of incubated solutions were taken for electron microscopic observations after 3-5 days. Amyloid fibril formation was verified by electron microscopic images of negatively stained samples.

For electron microscopic analysis, a 5 μL sample of the amyloid fibril was placed on a carbon coated Foamvar 200 mesh copper grid. The sample was allowed to stand for 15-30 sec and any excess solution was removed by wicking away. The samples were negatively stained with 2% (w/v) uranyl acetate. The excess was wicked away and allowed to dry. The samples were then visualized under a JEOL 1200EX transmission electron microscope operating at 80 Kev. The magnification ranged from 12,OOOX to 100,000X. Incubated solutions were lyophilized for solid-state NMR measurements.

Solid-state NMR samples contained 3-10 mg of peptide.

As is evident form the foregoing, the present invention provides novel and non-obvious materials and methods for the treatment, for example, of patients having symptoms of Alzheimer's disease and other diseases associated with amyloid plaque formation as well as patient's at risk for these diseases.

We claim:

1. An isolated protein comprising an amino acid sequence selected from the group consisting of SEQ ID NO: 1 and SEQ ID NOs:5-39.

2. The protein of claim 1, further comprising a protease-resistant protecting group.

3. An isolated protein comprising an amino acid sequence selected from the group consisting of SEQ ID NO:2 and SEQ ID NOs:40-74.

4. The protein of claim 3, further comprising a protease-resistant protecting group.

5. An isolated protein comprising an amino acid sequence selected from the group consisting of SEQ ID NO:3 and SEQ ID NOs:75-86.

6. The protein of claim 5, further comprising a protease-resistant protecting group.

7. An isolated protein comprising an amino acid selected from the group consisting of SEQ ID NO:4 and SEQ ID NOs:87-98.

8. The protein of claim 7, further comprising a protease-resistant protecting group.

9. An isolated amyloid fibril inhibitor protein comprising a repeating core sequence having an amino terminus and a carboxyl terminus, wherein said core sequence comprises a first and second protein binding amino acid residue and at least one polar amino acid residue.

10. The protein of claim 9, wherein said first binding residue is selected from the group consisting of glycine, alanine and serine.

11. The protein of claim 9, wherein said second binding residue is selected from the group consisting of phenylalanine, tyrosine, tryptophan, valine, leucine and isoleucine.

12. The protein of claim 9, wherein said polar residue is selected from the group consisting of histidine, lysine, arginine, aspartate, glutamate, asparagine, and glutamine.

13. The protein of claim 9, wherein said core sequence repeats between 1 to 5 times.

14. The protein of claim 9, wherein said core sequence further comprises at least one additional amino acid at said amino terminus.

15. The protein of claim 9, wherein said core sequence further comprises at least one additional amino acid at said carboxyl terminus.

16. The protein of claim 9, wherein said core sequence further comprises at least one additional amino acid at said carboxyl terminus and said amino terminus.

17. The protein of claim 9, further comprising a protease-resistant protecting group.

18. The protein of claim 9, wherein said core sequence is selected from the group consisting of SEQ ID NO:99-SEQ ID NO:146.

19. A composition comprising an Aβ₁₄₂ fibril comprising at least one phenylalanine amino acid residue and an isolated protein comprising a carboxyl terminus selected from the group consisting of SEQ ID NOs:1-4 and 19, wherein said protein carboxyl terminus binds to said fibril phenylalanine residue.

20. The composition of claim 19, wherein said protein has amyloid plaque dissolving activity.

21. The composition of claim 19, wherein said protein comprises a D-amino acid.

22. The composition of claim 19, wherein said protein comprises a protease resistance group.

23. The composition of claim 19, wherein said protein comprises a protecting group.

24. The composition of claim 19, wherein said protein is N-terminally acetylated and C-terminally amidated.

25. The composition of claim 19, wherein said protein comprises at least five additional N-terminal amino acids and C-terminal amino acids.

26. A method, comprising:
a) providing;
i) an amyloid fibril inhibitor protein comprising a core sequence, wherein said core sequence comprises a first and second protein binding amino acid residue and at least one polar amino acid residue;
ii) a patient comprising a peptide fibril;
b) contacting said inhibitor protein to said fibril under conditions such that said fibril depolymerizes.

27. The method of claim 26, wherein said fibril results from Alzheimer's disease.

28. The method of claim 26, wherein said fibril results from Parkinson's disease.

29. A method, comprising:
a) providing
i) an amyloid fibril inhibitor protein comprising a core sequence, wherein said core sequence comprises a first and second protein binding amino acid residue and at least one polar amino acid residue;
ii) a patient at risk for peptide fibril formation;
c) administering said inhibitor protein to said patient under conditions such that prevent polymerization of said peptide fibril in said patient.

30. A method of treating Alzheimer's disease, comprising:
(a) providing:
(i) a subject having symptoms of Alzheimer's disease and,
(ii) a composition of matter comprising a protein having a core sequence, wherein said core sequence comprises a first and second protein binding amino acid residue and at least one polar amino acid residue;
(b) administering said composition to said subject until said symptoms are reduced.

31. The method of claim 30, wherein said protein comprises from between four and five hundred amino acids.

32. The method of claim 30, wherein said protein is selected from a group consisting of SEQ ID NO: 1, SEQ ID NO:2, SEQ ID NO:3, SEQ ID NO:4 and variants thereof.

33. The method of claim 30, wherein said amino acid terminus is blocked with an acetyl group and said carboxyl terminus is blocked with an acetate group.

34. The method of claim 30, wherein said protein further comprises at least one non-naturally occurring amino acid.

35. The method of claim 30, wherein said protein further comprises at least one 1½ D-amino acid.


SEQUENCE LISTING (166 sequences)

SEQ ID NO: 1 - protein, 8 aa, Artificial Sequence
  Synthetic
RGTFEGKF

SEQ ID NO: 2 - protein, 8 aa, Artificial Sequence
  Synthetic
RFTGEFKG

SEQ ID NO: 3 - protein, 6 aa, Artificial Sequence
  Synthetic
RFTGEF

SEQ ID NO: 4 - protein, 6 aa, Artificial Sequence
  Synthetic
RFTAEF

SEQ ID NO: 5 - protein, 8 aa, Artificial Sequence
  Synthetic
RGTFEGKL

SEQ ID NO: 6 - protein, 8 aa, Artificial Sequence
  Synthetic
RGTFEGKM

SEQ ID NO: 7 - protein, 8 aa, Artificial Sequence
  Synthetic
RGTFEGKI

SEQ ID NO: 8 - protein, 8 aa, Artificial Sequence
  Synthetic
RGTFEGKV

SEQ ID NO: 9 - protein, 8 aa, Artificial Sequence
  Synthetic
RATFEGKF

SEQ ID NO: 10 - protein, 8 aa, Artificial Sequence
  Synthetic
RATFEGKL

SEQ ID NO: 11 - protein, 8 aa, Artificial Sequence
  Synthetic
RATFEGKM

SEQ ID NO: 12 - protein, 8 aa, Artificial Sequence
  Synthetic
RATFEGKI

SEQ ID NO: 13 - protein, 8 aa, Artificial Sequence
  Synthetic
RATFEGKV

SEQ ID NO: 14 - protein, 8 aa, Artificial Sequence
  Synthetic
RGTFEAKF

SEQ ID NO: 15 - protein, 8 aa, Artificial Sequence
  Synthetic
RGTFEAKL

SEQ ID NO: 16 - protein, 8 aa, Artificial Sequence
  Synthetic
RGTFEAKM

SEQ ID NO: 17 - protein, 8 aa, Artificial Sequence
  Synthetic
RGTFEAKI

SEQ ID NO: 18 - protein, 8 aa, Artificial Sequence
  Synthetic
RGTFEAKV

SEQ ID NO: 19 - protein, 8 aa, Artificial Sequence
  Synthetic
RATFEAKF

SEQ ID NO: 20 - protein, 8 aa, Artificial Sequence
  Synthetic
RATFEAKL

SEQ ID NO: 21 - protein, 8 aa, Artificial Sequence
  Synthetic
RATFEAKM

SEQ ID NO: 22 - protein, 8 aa, Artificial Sequence
  Synthetic
RATFEAKI

SEQ ID NO: 23 - protein, 8 aa, Artificial Sequence
  Synthetic
RATFEAKV

SEQ ID NO: 24 - protein, 8 aa, Artificial Sequence
  Synthetic
RATLEAKF

SEQ ID NO: 25 - protein, 8 aa, Artificial Sequence
  Synthetic
RATMEAKF

SEQ ID NO: 26 - protein, 8 aa, Artificial Sequence
  Synthetic
RATIEAKF

SEQ ID NO: 27 - protein, 8 aa, Artificial Sequence
  Synthetic
RATVEAKF

SEQ ID NO: 28 - protein, 8 aa, Artificial Sequence
  Synthetic
RGTWEGKW

SEQ ID NO: 29 - protein, 8 aa, Artificial Sequence
  Synthetic
RGTYEGKY

SEQ ID NO: 30 - protein, 8 aa, Artificial Sequence
  Synthetic
RGTWEGKF

SEQ ID NO: 31 - protein, 8 aa, Artificial Sequence
  Synthetic
RGTFEGKW

SEQ ID NO: 32 - protein, 8 aa, Artificial Sequence
  Synthetic
RGTYEGKF

SEQ ID NO: 33 - protein, 8 aa, Artificial Sequence
  Synthetic
RGTFEGKY

SEQ ID NO: 34 - protein, 8 aa, Artificial Sequence
  Synthetic
RATFEAKY

SEQ ID NO: 35 - protein, 8 aa, Artificial Sequence
  Synthetic
RATYEAKF

SEQ ID NO: 36 - protein, 8 aa, Artificial Sequence
  Synthetic
RATFEAKW

SEQ ID NO: 37 - protein, 8 aa, Artificial Sequence
  Synthetic
RATWEAKF

SEQ ID NO: 38 - protein, 8 aa, Artificial Sequence
  Synthetic
RATWEAKW

SEQ ID NO: 39 - protein, 8 aa, Artificial Sequence
  Synthetic
RATYEAKY

SEQ ID NO: 40 - protein, 8 aa, Artificial Sequence
  Synthetic
RLTGEFKG

SEQ ID NO: 41 - protein, 8 aa, Artificial Sequence
  Synthetic
RMTGEFKG

SEQ ID NO: 42 - protein, 8 aa, Artificial Sequence
  Synthetic
RITGEFKG

SEQ ID NO: 43 - protein, 8 aa, Artificial Sequence
  Synthetic
RVTGEFKG

SEQ ID NO: 44 - protein, 8 aa, Artificial Sequence
  Synthetic
RFTAEFKG

SEQ ID NO: 45 - protein, 8 aa, Artificial Sequence
  Synthetic
RFTAELKG

SEQ ID NO: 46 - protein, 8 aa, Artificial Sequence
  Synthetic
RFTAEMKG

SEQ ID NO: 47 - protein, 8 aa, Artificial Sequence
  Synthetic
RFTAEIKG

SEQ ID NO: 48 - protein, 8 aa, Artificial Sequence
  Synthetic
RFTAEVKG

SEQ ID NO: 49 - protein, 8 aa, Artificial Sequence
  Synthetic
RFTGEFKA

SEQ ID NO: 50 - protein, 8 aa, Artificial Sequence
  Synthetic
RLTGEFKA

SEQ ID NO: 51 - protein, 8 aa, Artificial Sequence
  Synthetic
RMTGEFKA

SEQ ID NO: 52 - protein, 8 aa, Artificial Sequence
  Synthetic
RITGEFKA

SEQ ID NO: 53 - protein, 8 aa, Artificial Sequence
  Synthetic
RVTGEFKA

SEQ ID NO: 54 - protein, 8 aa, Artificial Sequence
  Synthetic
RFTAEFKA

SEQ ID NO: 55 - protein, 8 aa, Artificial Sequence
  Synthetic
RFTAELKA

SEQ ID NO: 56 - protein, 8 aa, Artificial Sequence
  Synthetic
RFTAEMKA

SEQ ID NO: 57 - protein, 8 aa, Artificial Sequence
  Synthetic
RFTAEIKA

SEQ ID NO: 58 - protein, 8 aa, Artificial Sequence
  Synthetic
RFTAEVKA

SEQ ID NO: 59 - protein, 8 aa, Artificial Sequence
  Synthetic
RLTAEFKA

SEQ ID NO: 60 - protein, 8 aa, Artificial Sequence
  Synthetic
RMTAEFKA

SEQ ID NO: 61 - protein, 8 aa, Artificial Sequence
  Synthetic
RITAEFKA

SEQ ID NO: 62 - protein, 8 aa, Artificial Sequence
  Synthetic
RVTAEFKA

SEQ ID NO: 63 - protein, 8 aa, Artificial Sequence
  Synthetic
RWTGEWKG

SEQ ID NO: 64 - protein, 8 aa, Artificial Sequence
  Synthetic
RYTGEYKG

SEQ ID NO: 65 - protein, 8 aa, Artificial Sequence
  Synthetic
RWTGEFKG

SEQ ID NO: 66 - protein, 8 aa, Artificial Sequence
  Synthetic
RFTGEWKG

SEQ ID NO: 67 - protein, 8 aa, Artificial Sequence
  Synthetic
RYTGEFKG

SEQ ID NO: 68 - protein, 8 aa, Artificial Sequence
  Synthetic
RFTGEYKG

SEQ ID NO: 69 - protein, 8 aa, Artificial Sequence
  Synthetic
RFTAEYKA

SEQ ID NO: 70 - protein, 8 aa, Artificial Sequence
  Synthetic
RYTAEFKA

SEQ ID NO: 71 - protein, 8 aa, Artificial Sequence
  Synthetic
RFTAEWKA

SEQ ID NO: 72 - protein, 8 aa, Artificial Sequence
  Synthetic
RWTAEFKA

SEQ ID NO: 73 - protein, 8 aa, Artificial Sequence
  Synthetic
RWTAEWKA

SEQ ID NO: 74 - protein, 8 aa, Artificial Sequence
  Synthetic
RYTAEYKA

SEQ ID NO: 75 - protein, 7 aa, Artificial Sequence
  Synthetic
RVFTGEF

SEQ ID NO: 76 - protein, 6 aa, Artificial Sequence
  Synthetic
RLTGEF

SEQ ID NO: 77 - protein, 6 aa, Artificial Sequence
  Synthetic
RMTGEF

SEQ ID NO: 78 - protein, 6 aa, Artificial Sequence
  Synthetic
RITGEF

SEQ ID NO: 79 - protein, 6 aa, Artificial Sequence
  Synthetic
RWTGEF

SEQ ID NO: 80 - protein, 6 aa, Artificial Sequence
  Synthetic
RYTGEF

SEQ ID NO: 81 - protein, 6 aa, Artificial Sequence
  Synthetic
RFTGEV

SEQ ID NO: 82 - protein, 6 aa, Artificial Sequence
  Synthetic
RFTGEL

SEQ ID NO: 83 - protein, 6 aa, Artificial Sequence
  Synthetic
RFTGEM

SEQ ID NO: 84 - protein, 6 aa, Artificial Sequence
  Synthetic
RFTGEI

SEQ ID NO: 85 - protein, 6 aa, Artificial Sequence
  Synthetic
RFTGEW

SEQ ID NO: 86 - protein, 6 aa, Artificial Sequence
  Synthetic
RFTGEY

SEQ ID NO: 87 - protein, 7 aa, Artificial Sequence
  Synthetic
RVFTAEF

SEQ ID NO: 88 - protein, 6 aa, Artificial Sequence
  Synthetic
RLTAEF

SEQ ID NO: 89 - protein, 6 aa, Artificial Sequence
  Synthetic
RMTAEF

SEQ ID NO: 90 - protein, 6 aa, Artificial Sequence
  Synthetic
RITAEF

SEQ ID NO: 91 - protein, 6 aa, Artificial Sequence
  Synthetic
RWTAEF

SEQ ID NO: 92 - protein, 6 aa, Artificial Sequence
  Synthetic
RYTAEF

SEQ ID NO: 93 - protein, 6 aa, Artificial Sequence
  Synthetic
RFTAEV

SEQ ID NO: 94 - protein, 6 aa, Artificial Sequence
  Synthetic
RFTAEL

SEQ ID NO: 95 - protein, 6 aa, Artificial Sequence
  Synthetic
RFTAEM

SEQ ID NO: 96 - protein, 6 aa, Artificial Sequence
  Synthetic
RFTAEI

SEQ ID NO: 97 - protein, 6 aa, Artificial Sequence
  Synthetic
RFTAEW

SEQ ID NO: 98 - protein, 6 aa, Artificial Sequence
  Synthetic
RFTAEY

SEQ ID NO: 99 - protein, 3 aa, Artificial Sequence
  Synthetic
FXG

SEQ ID NO: 100 - protein, 3 aa, Artificial Sequence
  Synthetic
FXA

SEQ ID NO: 101 - protein, 3 aa, Artificial Sequence
  Synthetic
FXS

SEQ ID NO: 102 - protein, 3 aa, Artificial Sequence
  Synthetic
WXG

SEQ ID NO: 103 - protein, 3 aa, Artificial Sequence
  Synthetic
YXG

SEQ ID NO: 104 - protein, 3 aa, Artificial Sequence
  Synthetic
WXA

SEQ ID NO: 105 - protein, 3 aa, Artificial Sequence
  Synthetic
YXA

SEQ ID NO: 106 - protein, 3 aa, Artificial Sequence
  Synthetic
FXG

SEQ ID NO: 107 - protein, 6 aa, Artificial Sequence
  Synthetic
XFXGXG

SEQ ID NO: 108 - protein, 6 aa, Artificial Sequence
  Synthetic
XFXGXA

SEQ ID NO: 109 - protein, 6 aa, Artificial Sequence
  Synthetic
XFXGXS

SEQ ID NO: 110 - protein, 6 aa, Artificial Sequence
  Synthetic
XFXAXA

SEQ ID NO: 111 - protein, 6 aa, Artificial Sequence
  Synthetic
XFXAXG

SEQ ID NO: 112 - protein, 6 aa, Artificial Sequence
  Synthetic
XFXSXG

SEQ ID NO: 113 - protein, 6 aa, Artificial Sequence
  Synthetic
XYXGXG

SEQ ID NO: 114 - protein, 6 aa, Artificial Sequence
  Synthetic
XWXGXG

SEQ ID NO: 115 - protein, 6 aa, Artificial Sequence
  Synthetic
XFXFXG

SEQ ID NO: 116 - protein, 6 aa, Artificial Sequence
  Synthetic
XFXFXA

SEQ ID NO: 117 - protein, 6 aa, Artificial Sequence
  Synthetic
XFXFXS

SEQ ID NO: 118 - protein, 6 aa, Artificial Sequence
  Synthetic
XYXFXG

SEQ ID NO: 119 - protein, 6 aa, Artificial Sequence
  Synthetic
XWXFXG

SEQ ID NO: 120 - protein, 6 aa, Artificial Sequence
  Synthetic
XFXWXG

SEQ ID NO: 121 - protein, 6 aa, Artificial Sequence
  Synthetic
XFXYXG

SEQ ID NO: 122 - protein, 6 aa, Artificial Sequence
  Synthetic
XYXFXA

SEQ ID NO: 123 - protein, 4 aa, Artificial Sequence
  Synthetic
XGXF

SEQ ID NO: 124 - protein, 4 aa, Artificial Sequence
  Synthetic
XAXF

SEQ ID NO: 125 - protein, 4 aa, Artificial Sequence
  Synthetic
XSXF

SEQ ID NO: 126 - protein, 4 aa, Artificial Sequence
  Synthetic
XGXW

SEQ ID NO: 127 - protein, 4 aa, Artificial Sequence
  Synthetic
XGXY

SEQ ID NO: 128 - protein, 4 aa, Artificial Sequence
  Synthetic
XAXW

SEQ ID NO: 129 - protein, 4 aa, Artificial Sequence
  Synthetic
XAXY

SEQ ID NO: 130 - protein, 4 aa, Artificial Sequence
  Synthetic
XSXW

SEQ ID NO: 131 - protein, 6 aa, Artificial Sequence
  Synthetic
XGXGXF

SEQ ID NO: 132 - protein, 6 aa, Artificial Sequence
  Synthetic
XAXGXF

SEQ ID NO: 133 - protein, 6 aa, Artificial Sequence
  Synthetic
XSXGXF

SEQ ID NO: 134 - protein, 6 aa, Artificial Sequence
  Synthetic
XAXAXF

SEQ ID NO: 135 - protein, 6 aa, Artificial Sequence
  Synthetic
XGXAXF

SEQ ID NO: 136 - protein, 6 aa, Artificial Sequence
  Synthetic
XGXSXF

SEQ ID NO: 137 - protein, 6 aa, Artificial Sequence
  Synthetic
XGXGXY

SEQ ID NO: 138 - protein, 6 aa, Artificial Sequence
  Synthetic
XGXGXW

SEQ ID NO: 139 - protein, 6 aa, Artificial Sequence
  Synthetic
XGXFXF

SEQ ID NO: 140 - protein, 6 aa, Artificial Sequence
  Synthetic
XAXFXF

SEQ ID NO: 141 - protein, 6 aa, Artificial Sequence
  Synthetic
XSXFXF

SEQ ID NO: 142 - protein, 6 aa, Artificial Sequence
  Synthetic
XGXFXF

SEQ ID NO: 143 - protein, 6 aa, Artificial Sequence
  Synthetic
XGXFXW

SEQ ID NO: 144 - protein, 6 aa, Artificial Sequence
  Synthetic
XGXWXF

SEQ ID NO: 145 - protein, 6 aa, Artificial Sequence
  Synthetic
XGXFXY

SEQ ID NO: 146 - protein, 6 aa, Artificial Sequence
  Synthetic
XGXFXY

SEQ ID NO: 147 - protein, 3 aa, Artificial Sequence
  Synthetic
XXX

SEQ ID NO: 148 - protein, 6 aa, Artificial Sequence
  Synthetic
XXXXXX

SEQ ID NO: 149 - protein, 6 aa, Artificial Sequence
  Synthetic
XXXXXX

SEQ ID NO: 150 - protein, 4 aa, Artificial Sequence
  Synthetic
XXXX

SEQ ID NO: 151 - protein, 6 aa, Artificial Sequence
  Synthetic
XXXXXX

SEQ ID NO: 152 - protein, 6 aa, Artificial Sequence
  Synthetic
XXXXXX

SEQ ID NO: 153 - protein, 10 aa, Artificial Sequence
  Synthetic
RFTGEFKGTF

SEQ ID NO: 154 - protein, 10 aa, Artificial Sequence
  Synthetic
RGTFEGKFTG

SEQ ID NO: 155 - protein, 10 aa, Artificial Sequence
  Synthetic
XXXXXXXXXX

SEQ ID NO: 156 - protein, 10 aa, Artificial Sequence
  Synthetic
XRGTFQGKFX

SEQ ID NO: 157 - protein, 42 aa, Artificial Sequence
  Synthetic
DEAFRHDSGYEVHHQKLVFFAEDVGSNKGAIIGLMVGGVVIA

SEQ ID NO: 158 - protein, 17 aa, Artificial Sequence
  Synthetic
EPEITLIIFGVMAGVIG

SEQ ID NO: 159 - protein, 18 aa, Artificial Sequence
  Synthetic
AGAVVGGLGGYMLGSAMS

SEQ ID NO: 160 - protein, 5 aa, Artificial Sequence
  Synthetic
LPFFD

SEQ ID NO: 161 - protein, 7 aa, Artificial Sequence
  Synthetic
GXFXGXF

SEQ ID NO: 162 - protein, 8 aa, Artificial Sequence
  Synthetic
RXTXEXKX

SEQ ID NO: 163 - protein, 29 aa, Artificial Sequence
  Synthetic
EPEITLIIFGVMAGVIGTILLISYGIRRL

SEQ ID NO: 164 - protein, 8 aa, Artificial Sequence
  Synthetic
RGTFQGXF

SEQ ID NO: 165 - protein, 53 aa, Artificial Sequence
  Synthetic
DEAFRHDSGYEVHHQKLVFFAEDVGSNKGAIIGLMVGGVVIATVIVITLVMLK

SEQ ID NO: 166 - protein, 8 aa, Artificial Sequence
  Synthetic
RGTFQGKF